(19) United States
(12) Patent Application Publication
(10) Pub. No.: US 2024/0374863 A1
(43) Pub. Date: Nov. 14, 2024

(54) A computing system for treatment of a neural disorder

(71) Applicant: Ababax Health GmbH, Berlin (DE)

(72) Inventors: David Aagten-Murphy, Berlin (DE); Hanni Kiiski, Berlin (DE); Liam Doherty, Berlin (DE)

(21) Appl. No.: 18/692,534

(22) PCT Filed: Sep. 15, 2022
(86) PCT No.: PCT/EP2022/075662

§ 371 Date: Mar. 15, 2024

(30) Foreign Application Priority Data
Sep. 15, 2021 (LU) 500654

(51) Int. Cl. A61M 21/02; A61M 21/00; G16H 20/70

(52) U.S. Cl. CPC A61M 21/02; G16H 20/70; A61M 2021/0016; A61M 2021/0022; A61M 2021/0027; A61M 2021/0044; A61M 2205/3303; A61M 2205/3553; A61M 2205/3561; A61M 2205/505; A61M 2205/507; A61M 2205/581; A61M 2205/582; A61M 2205/583; A61M 2205/588; A61M 2209/088; A61M 2230/10

(57) ABSTRACT

A computing system for treatment of a neural disorder of a user. The system comprises a processor, a memory, and an output device. The computing system further comprises an oscillator module adapted to run on the processor, thereby interacting with the output device to produce rhythmic sensory stimuli, perceivable by senses of the user, at at least one predetermined frequency to induce oscillations in a brain of the user and thereby to enhance neural activity of a neural circuit associated with the processing of a task. The computing system further comprises a content module stored in the memory interacting with the output device to produce content to engage the user. A method for treatment of a neural disorder in a user comprising providing rhythmic sensory stimuli, to induce oscillations in a brain of user to enhance neural activity of a neural circuit associated with the processing of a task.

### BACKGROUND OF THE INVENTION

### Field of the Invention

This application claims priority of the Luxembourg Patent Application number LU500654, filed on 15 Sep. 2021. The entire disclosure of the Luxembourg Patent Application number LU500654 is hereby incorporated herein by reference.

The present invention relates to devices, systems, and methods for treating mental disorders, such as disorders relating to depression, anxiety, obesity, and pain, and for enhancing immunity.

### Brief Description of the Related Art

It is known that every task carried out in the mind (also termed perceptual and/or psychological and/or cognitive event) elicits a distinct pattern of neural activity in a brain. This includes both simple tasks, such as looking at an object or recognising a famous face, and much more complex tasks like solving a difficult puzzle, performing numerical operations, or remembering what happened long ago.

These distinct patterns of neural activity are associated with the brain's information processing of the specific perceptual, psychological, and/or cognitive event or task in question. These patterns of neural activity evolve, and the patterns change across time as the brain processes the information. The patterns evolve and change across the space of the brain as different regions and areas are involved with different processing steps of the information at different times.

For example, imagine a first scenario in which a user is shown a screen with two digits, one digit on the left and one digit on the right, and the user is tasked with pressing a key with either their left hand or their right hand depending on which one of the two digits represents a larger quantity. From the initial appearance of the stimuli with the two digits until the eventual response by the user, the major regions of activity in the brain will shift from visual areas (as the visual input is processed and the digits are recognised), towards frontal decision-making areas (as the decision is made regarding which one of the digits represents a larger quantity), before finally moving towards motor areas (to initiate the response, i.e., pressing the key). This evolving pattern of neural activity reflects the perceptual, psychological, and/or cognitive processing associated with the task given to the user.

The patterns of the neural activity related to a specific task are generally consistent, enabling the status of an individual's information processing at any specific point in time to be estimated by readouts of the neural activity (via EEG). Since these readouts reflect the perceptual, psychological, and/or cognitive processing of the user at a specific point in time, the readouts can be used to make predictions about the performance, decision, or action which the user is likely to initiate in response to the task or the stimuli in question—even before the user is aware that the users have made the decision or have begun to initiate the response themselves.

However, while generally consistent, the neural activity for the exact same task (or response to the stimuli) when repeated inevitably varies. The brain is a highly hierarchical, networked, and complex system and its complex dynamics are very susceptible to changes in the initial conditions. Even very small changes in the initial state of the brain can have profound consequences for how the pattern of the neural activity in the brain changes over time. This change in the initial state can result in subtle perturbations, in which certain stages of the processing take a longer or shorter time, resulting in slower or faster reaction times for a task. The change in the initial state can also result in more crucial perturbations in which the user arrives at a different decision and initiates an entirely different response to the stimuli.

Both types of results are intuitively familiar. Even when responding to the exact same prompt (or stimulus) and trying to be as fast as possible, the reaction times of users will vary, and it is found that the users respond faster or slower from trial to trial. The intuitive feeling of the user is one of being at times “more ready” or “less ready”. Similarly, the user will sometimes make the wrong response, especially when the user is trying to respond as fast as possible.

One of the reasons for the changes is that the user's brain state is constantly changing, and that the capacity of neurons in the brain to process a new event (i.e., perceptual, psychological, and/or cognitive event) at any given time will be influenced by the underlying brain state of the user at the time the event occurs. The brain state is, by default, always fluctuating on many different levels, from the neural (i.e., the individual neurons) to the circuitry (i.e., interconnected networks of neurons). Thus, even when the user is presented with identical tasks comprising identical stimuli, the user's perception of the stimuli and their subsequent response to the stimuli (be that their liking or disliking, the speed the users take to press a key, or any other stimulus derived response) will vary due to the differential initial states of their brain.

These changes in the brain state result from many factors within the complex system that is the human brain. Global changes in the brain state can be caused by cognitive or bodily processes, such as a shifting focus of attention, a changing of the goals of the task the user is performing, or the user experiencing fatigue. At the same time, local changes in the brain state such as cyclic firing from clusters of neurons or delays in neurons' firing due to recovery time between firings are also contributing to the user's brain state.

Many such changes in the brain state are inherently rhythmic or periodical when looked at across different time scales. Across the span of days, a user will go through periodical changes in alertness (during the day) and tiredness (during the night). Although there is some day-to-day variation, the rhythmic or periodical cycles repeat on average over a time span such as several days. Further periodical changes are found in bodily functions such as breathing (with inhalation and exhalation) or heartbeat.

If the user is demotivated, distracted, and fatigued, then the user's sensitivity and reaction times are likely to be slower compared to when the user is motivated, focused, and well rested. At any specific moment, the user will be at different stages of the multiple rhythmic cycles occurring in the brain and operating over different time scales. The sum or superposition of multiple rhythmic cycles contribute to the user's current brain state. The multiple rhythmic cycles or oscillations and their interactions produce brain states that either help or hinder the processing of external information, i.e., of a task or perceptual, psychological, and/or cognitive event.

The brain is organised hierarchically and, although the activity of the brain is often described at a specific level of this hierarchy, the processes in the brain are occurring (and interacting) at all levels of the hierarchy simultaneously. While an individual neuron may have its own periodic behaviour, the same individual neuron is influencing (and is influenced by) the activity of the circuit and larger region in which the individual neuron is situated. A useful analogy for this is to consider a crowd moving through a narrow street or passage as they exit from a stadium. Individuals in the crowd will all be moving at different speeds and in slightly different directions, each with their own rhythmic step, but the crowd altogether will also have a “collective” direction and a common rhythm to the collective movement. An individual in the crowd could push through and walk against the majority of the rest of the crowd, which, however, would require a lot more energy and determination than simply moving in the same direction as the crowd. Similarly, the broader oscillations in a neural region will influence—but not necessarily dominate—the activity of the circuits and neurons from which the neural regions are assembled.

In addition to the neural activity that occurs during the processing of a task or the perceptual, psychological and/or cognitive event, the patterns of the neural activity that directly precede the task are also important as these preceding patterns define the initial brain state under which the task or perceptual, psychological and/or cognitive event is processed.

This initial brain state influences how the incoming sensory information is processed by the perceptual system and in turn alters the neural activity associated with processing the task or the perceptual, psychological and/or cognitive event. The influence of the initial brain state can be complementary to the processing of the incoming sensory information, or the initial brain state can interfere with its processing, depending on the task or the perceptual, psychological and/or cognitive event and the status of the initial brain state. This means that, with the right initial brain state for the specific task or perceptual, psychological and/or cognitive event, the user's processing can be enhanced.

The overall brain state of an individual is a function of their neural activity occurring at all levels of the brain's hierarchy at any given time. One particularly meaningful level of this hierarchy are large-scale neural oscillatory activity or oscillations that occur when large groups of neurons in specific regions of the brain synchronise their firing patterns to a particular temporal frequency.

These brain rhythms oscillate between high and low states of neural excitability, reflecting synchronized (increased coordinated neural activity) and desynchronized (lower coordinated neural activity), respectively. They are characterised in terms of their rhythmic qualities (e.g., frequency), strength (i.e., power or amplitude) and timing (i.e., phase). Different oscillatory rhythms are linked to distinct cognitive processes and aspects of information processing at large.

The oscillatory patterns that have been observed to occur in the human brain and are associated with different cognitive processes can have frequencies in a frequency range of between 0 and 300 Hz. The frequency range comprises five frequency bands (that differ in the number of oscillations/cycles occurring per 1 second, Hz): delta band (0.5-4 Hz), theta band (4-8 Hz), alpha band (8-13 Hz), beta band (13-30 Hz) and gamma band (30-200 Hz). These oscillatory patterns arise from the electrical activity of a population of neurons. The oscillations can be synchronized when groups of neurons from different brain areas have coordinated firing patterns, or the oscillations can be desynchronized when neuron activities are in opposite phases and cause inhibition. Depending on the role and function of the neuronal population in question, these synchronised activities can have profound influences on how a given task or the perceptual, psychological and/or cognitive event is processed and responded to by an individual.

The oscillatory patterns can arise ‘endogenously’, without a specific external triggering event, from the co-ordinated activity of different neuronal populations, or the oscillatory patterns can occur in response to an external stimulus or an event or a task. The oscillatory patterns are thought to be closely interlinked with anatomical architecture of the brain. In general, higher frequency oscillations are confined to a small neuronal space and engage local networks related to bottom-up processing, whereas slow rhythms (lower frequency oscillations) synchronize very large networks and can bind together specific assemblies by the appropriate timing of higher frequency localized oscillations, and this long-range integrative processing is related to top-down control processes.

Slow rhythms like theta (4-8 Hz) have been associated with encoding new, episodic information whereas alpha rhythms (9-13 Hz) have been linked with inhibitory processing and long-term memory retrieval. Faster rhythms like gamma (>30 Hz), on the other hand, have been implicated in feed-forward processing and neural plasticity, among other functional roles.

These endogenous oscillatory patterns have been found to be relevant to a person's experience and behaviour. For example, the strength and synchrony of neural oscillations occurring within the visual cortex has been shown to index the likelihood of perceiving a visual stimulus (Thut, 2006) whereas strength and synchrony of neural oscillations during memory consolidation can predict later recall (Brokaw et al., 2016). Even when shown the exact same stimulus and asked to make the exact same response, variation for the current response can be predicted from the variation in the neural activity (de Graaf et al., 2013).

Performance on many cognitive and perceptual tasks has been shown to be highly dependent on the state of the brain prior to, and during, the onset of the task/stimulus themselves. The level and timing of neural activity in visual cortex—and particular to the phase and strength of ongoing oscillatory activity of different frequency bands—immediately prior to stimulus onset, for instance, can predict visual perception, and memory performance.

The US patent application no. 2020/155852 A1 discloses direct stimulation of nerve tissue of interest, such as brain tissue, spinal cord tissue, dorsal root ganglion tissue, by means of stimulation electrodes using nested stimulation waveforms. The application of nested stimulation waveforms to nerve tissue of interest aims to entrain intrinsic neural oscillations. Parameters of the nested stimulation waveforms may be set to achieve entrainment of the neural oscillations in connection with at least one of a sensory, motor, and cognitive event, e.g., in the gamma wave frequency band.

The international patent application no. WO 2014/006596 A1 discloses a sound therapy system comprising loudspeakers and a computer-based software application module for creating structured sound. In one embodiment, neural oscillation frequencies are added to a sound treatment signal. The neural oscillations are used to induce, via whole body exposure brain entrainment, various states in the treated subject such as slow-wave sleep (delta waves), meditative or sleeping states (theta waves), or wakeful states (alpha waves).

Light therapy has been reported to be beneficial for multiple medical conditions, including depression and sleeping disorders. In particular, visual exposure of rats to green light has been shown to produce antinociceptive (i.e., pain-sensation-inhibiting) and antihyperalgesic (increased-pain-sensitivity relieving) effects.

It is not fully understood whether and/or how activation of the visual pathway by exposure to green light mediates the antinociception effects. Opaque contact lenses that block penetration and/or transmission of any light abolish these green-light-mediated effects in rats. Mechanistic neural links between the optic nerve and the periaqueductal grey (PAG), have been described in rat experiments, where electrical stimulation of the optic nerve led to activity changes in the PAG. The PAG is a region of the brain which is known to modulate pain.

It has been a topic of research efforts whether analgesic (pain-relieving) effects of the green light are mediated by descending, opioid-sensitive pain inhibition. This potential mechanism of descending, opioid sensitive pain inhibition involves mu-opioid receptors and descending pain inhibitory pathways from the rostral ventromedial medulla (RVM) receiving input from the PAG. It is also known that increased spinal cord expression of mRNA of enkephalins, which are endogenous opioids, is involved in pain inhibition. It has moreover been shown in support of the presence of the potential mechanism that antinociceptive (pain-relieving) effects of green light are suppressed by administration of naloxone, an opioid receptor antagonist.

In humans, visual exposure to green light (1-2 hours per day for 10 weeks) has been shown to significantly reduce the number of headache days as well as intensity and duration of the headache attacks in patients with episodic migraine or chronic migraine. Following the same green light exposure protocol, fibromyalgia patients reported a significant reduction in pain intensity. Regarding the involvement of the opioid system, a PET (positron emission tomography) study in humans reported a functional link between direct deep brain stimulation of PAG and the release of endogenous opioids leading to analgesia.

Oscillations in the alpha range (or alpha oscillations) in the frequency band of 8-13 Hz are known to modulate somatosensation via inhibitory descending cortical projections (top-down) and changes in the power of alpha-band activity have been directly associated with pain perception.

Alpha oscillations drive the inhibition of ascending thalamocortical GABAergic synapses which transmit pain signals. Decreases in the power of alpha oscillations reflecting a reduced synchronization of neural ensembles, have been linked to enhanced perception of pain. In chronic pain and hypersensitivity, changes in the power and frequency of alpha oscillations have been associated with enhanced perception of noxious signals and pain intensity.

The alpha oscillations also influence the pain perception by modulating attention of the patient. Both acute and chronic pain is perceived as less intense if the patient's attention is shifted away from the pain. Thus, a distraction from the pain has, in itself, an analgesic effect. Alpha band activity thus may modulate pain salience, i.e., influence the likelihood of pain being perceived by the patient.

The alpha oscillations can be induced by using audio-visual stimulation, for example, by exposure to binaural beats and/or flashing lights such as LEDs mounted on a headband. Increased alpha power in cortical regions can be observed after only 4 minutes of stimulation at 10 Hz in both, healthy individuals as well as patients suffering chronic pain. This increase in the alpha power has been linked to reduced perception of acute pain. However, the mechanism linking alpha power and decreased pain perception is still not fully understood. It is furthermore not understood whether induction of alpha power enables reducing chronic pain significantly and/or persistently.

International patent application WO 2016/205669 A2 (Beth Israel Deaconess Medical Center) discloses a light source configured to emit visible radiation in the green region of the visible spectrum (i.e., a wavelength range between 510 nm and 550 nm). Furthermore, an eyeglass lens is disclosed that includes an optical filter that filters a portion of the visible light spectrum. The method and apparatus are used to aid users experiencing photophobia.

International patent application WO 2017/210498 A1 (University of Arizona) discloses the administration of blue or green spectrum light to the retina of a user exhibiting pain under conditions. The light has a wavelength of approximately 450 to 570 nm. A contact lens, eyeglasses, and goggles, that filter light outside said wavelength range, are disclosed. The patent application suggests that the method and system can be used for managing and minimizing chronic pain.

The US patent U.S. Pat. No. 10,962,789 B1 (Percept Technologies Inc.) discloses a digital eyewear with a frame, lenses, sensors for patient and ambient parameters, and a control element. The lenses may be configured to control passage or emission of light, e.g., blue light in the range of 450-490 nm or green light in the range of 520 to 560 nm, into the eyes of the user.

The US patent applications 2002/0156508 A1 and 2002/0044254 A1 (Saathoff Enterprises Inc.) discloses a contact lens or eyeglass lens that filter a portion of the ambient light.

The international patent application with the publication number WO 2019/226656 A1 (Sana Health Inc.) discloses a method of managing pain by administering a visual, auditory, and/or tactile stimulus to a patient by means of a headset comprising one or more light sources, sound sources, and bone conduction transducers. The system may comprise to measure parameters such as heart rate, heart rate variability, body temperature, or blood pressure.

The US patent application 2019/0200920 A1 (Tamade Inc.) discloses a biofeedback virtual reality (VR) system that monitors one or more physiological parameters of a user while presenting the user with an immersive VR environment. The system comprises a display, a software and computing system, a controller, and one or more physiological sensors.

The international patent application WO 2018/106839 A2 discloses a system for the treatment of migraine, headache, and other pain syndromes by means of auditory and visual stimuli. A visual indicator illuminates a portion of a treatment device with green light, which can have a therapeutic effect of relieving, e.g., migraine. A pressure-based ear stimulation modality (alone or coupled with sounds and/or air or bone conduction vibration) is configured to cycle at a selected frequency, such as about 10 Hz to facilitate transitional brain states.

Obesity is in most cases the result of altered eating behaviors, such as an increased intake of high-caloric and energy-dense food and drinks, as well as a sedentary work- and lifestyle. These behaviors can only be changed in a long-lasting manner by sustained changes in lifestyle.

Obesity is a behavioral addiction. It has been shown that obese individuals show structural and functional changes in brain networks involved in reward and salience processes. These networks span across diverse brain areas including the inferior prefrontal cortex, the orbitofrontal cortex, the ventral tegmental area, putamen and the nucleus accumbens. These brain areas display particular activation patterns in obese users that resemble those observed in addiction and substance abuse. Obesity thus shares features with addiction disorders and substance abuse disorders, which has led to the concept of the so-called food addiction.

It is not clear whether eating behaviors and weight loss can be influenced by non-invasive brain stimulation. A broad meta-analysis of studies concluded that no technique is known which achieves a significant reduction in body weight. Some techniques result in weak effects on weight loss and medium effects on cravings, inhibitory control and food consumption. The difficulties might be caused by the complexity and multifactorial nature of obesity. Furthermore, brain stimulation as a single treatment is regarded as not being effective for losing weight or subsequently maintaining a reduced weight in the long term. Brain stimulation might therefore have to be combined with other interventions.

Night eating syndrome (NES) is a condition in which patients suffer from insomnia with frequent night-time arousals from sleep and consumption of food during the night. In some cases, the patient's nocturnal consumption amounts to more than 25% of daily caloric intake. 1-2 percent of the population suffer from the syndrome. Due to nocturnal eating, patients have a higher risk for Diabetes mellitus and obesity, of which the prevalence in NES patients is 30.8-12% and 6-16%, respectively.

Due to common co-morbidities with depression, schizophrenia, and binge-eating, it is challenging to pinpoint precise causalities. Scientific evidence points to a shifted circadian rhythm due to wrong central clock cueing with eating and activity during the night. The shifting of the circadian rhythm may result in a delay of up to 5 hrs. Sleep timing is set by hormonal signals from the endogenous circadian clock in the human body. Disturbances of the circadian rhythm affect timing as well as quality of sleep.

Daylight is the most appropriate cue for our central clock, namely neurons of the Suprachiasmatic nucleus (SCN) of the hypothalamus, which in turn regulates organ clocks in the periphery (e.g., metabolism in liver, fat, muscle). Disturbances in circadian rhythm (wrong cueing due to, e.g., eating behaviour, working hours, stress, LED displays) are related to metabolic diseases (obesity, coronary heart disease, metabolic syndrome, diabetes II) by disturbances in melatonin release (phase shift and amount).

The photopigment melanopsin has fairly recently been found to be involved in entrainment of the circadian rhythm. Melanopsin is located in the inner retina of humans and other animals and is expressed in the intrinsically photosensitive retinal ganglion cells (ipRGCs). The ipRGCs respond to incoming light directly via melanopsin, as well as indirectly through signals from rods and cones. It is known that melanopsin is sensitive mainly to short wavelengths and, in particular, blue light having a wavelength in a range around 480 nm. The response of the ipRGCs to light is a non-image forming or non-visual photo-response and brings about circadian entrainment in many physiological or body functions. These functions include sleep/wake state (melatonin synthesis), pupil light reflex for regulation of retinal illumination, cognitive performance, mood, locomotor activity, memory, body temperature, etc. The ipRGCs indirect input via the SCN (Suprachiasmatic nucleus) regulates the light-sensitive suppression of melatonin production in the pineal gland.

Sleep normally involves a series of stages such as light sleep, deep sleep, or REM (rapid eye movement) sleep. These sleep stages are associated with brain waves that can be measured by means of EEG (electroencephalography). In a state of light sleep, the brain has been found to undergo theta waves. During deep sleep, on the other hand, the activity of the brain is characterized by delta waves.

A delta wave is a type of slow high-amplitude brain wave found in humans. Delta waves have a frequency from one to four hertz and are measured using an electroencephalogram (EEG). These brain waves are thought to emerge from the thalamus and are generally associated with slow-wave sleep during the third stage of sleep. This period of time during which delta waves occur is often known as deep sleep.

The patent application EP 3 524 308 A1 discloses inducing an individual to a sleep state using binaural acoustic tones. The method comprises positioning a sensor on the individual to detect electrical activity of the brain and changes in motion. The method comprises iteratively applying an adjusted binaural tone to adjust the current activity state of the individual's brain towards a target state to enable the individual to enter and maintain a restful sleep cycle naturally.

The patent application US 2022/047890 A1 discloses a light therapy system comprising a lighting assembly having a temporally controllable illuminance. The light therapy system further comprises a visual display. The lighting assembly and the visual display may be integrated into a single unit. Graphical output, such as a video or list of videos, may be presented to a patient in order to relax the patient by displaying images designed to invoke a particular mood.

The current mood of the user can influence both psychological and immunological responses to events, such as immunologically challenging events (ICEs) in which the immune system response is closely related to the user's health outcome. Examples of ICEs include infectious illness, vaccination, or surgical events. The user's mood at the time of the ICE influences the efficacy of the treatment and the likelihood to develop secondary problems, i.e. side-effects.

Research investigating associations between psychological experiences and immunity has largely focused on the effects of negative emotions such as stress and loneliness. It has been shown that “immune modulators” exist, such as physical activity, nutrition, mood, or stress. These modulators influence immune responses to, e.g., vaccination. More specifically, there is evidence of a direct link between greater positive affect and improved immune function. This link also affects the prevalence or strength of both placebo (positive consequences related to positive expectations) and nocebo (negative consequences related to negative expectations).

Stress can influence immune function by dysregulating humoral and cellular immune responses. This influence is due to interaction of the central nervous system (CNS), the endocrine system and the immune system. Secretory immunoglobulin A (sIgA), measured in, e.g., saliva, is an antibody that plays a role in the primary defense against viral and bacterial infections. Studies have shown that chronic psychological stress and increases in cortisol are associated with reductions in sIgA, which reductions are assumed to increase susceptibility to infections. The ability to ‘unwind’ after stressful events, to return to neuroendocrine baseline in a relatively short time, is thought to influence the total burden of stressors on an individual. Psychological stress has been associated with an increased rates of acute infectious respiratory illness in a dose-response manner, due to decreased host resistance.

Furthermore, psychological stress impacts negatively on vaccine responses. Distressed and anxious individuals display immune responses to vaccines that are delayed, substantially weaker and/or shorter-lived. In a study of the effect of mood on the effectiveness of an influenza vaccination, it was found that a positive mood increased IgG levels. Additionally, it was found that those with negative mood on the day of vaccination and throughout the mood tracking period had lower IgG levels.

Natural disasters, such as a pandemic, are longer-term stressors that have been associated with immune alterations (‘burnout’ at work, job strain, unemployment, and isolation). Stressors that are perceived as unpredictable and/or uncontrollable might continue to be associated with increased levels of stress hormones, even after repeated exposures. The Sars-Cov-2 pandemic and the implemented measures have dramatically increased stress in parts of the population, which could lead to an increase in immunological vulnerability as well as a decrease in the immunological responses to vaccination. Intervention research suggests that psychological and behavioral interventions can increase vaccine efficacy in the context of SARS-CoV-2 vaccination. Such interventions include techniques such as stress management, relaxation, cognitive behavioral therapy, and emotional disclosure.

The deliberate induction of positive mood prior to the experience of an ICE has been investigated as a potential method to enhance vaccination success. It has been found that various forms of positive mood induction (e.g. watching films, listening to music, reflecting on positive personal experiences) increase total sIgA levels in saliva. It is not known what long-term impacts on immunity the mood enhancements have.

Long-term improvements in positive affect and thus immune function are desirable, but interventions aimed at sustained changes in mood are often expensive, take time and are difficult to implement. For this reason, there is a need to beneficially manipulate the relationship between mood and immune function precisely at the right time.

The patent EA009693B1 is directed at a neurotherapeutic correction of a person's psychosomatic state by means of rhythmic light, sound and vibrotactile stimulation. The rhythmic stimulation has frequencies lying in the range between 4 and 16 Hz. Neural oscillations in the brain (“bioelectrical activity”) are associated with changes in cognitive processes as well as the psychoemotional, neurohormonal and immune status of a person. Directed oscillatory stimulation may result in planned physiological effects in, e.g., the immune system. A device includes a computer control unit, a signal conditioning unit, goggles with emitters, stereo headphones, a vibration plate with transducers. A course of neurotherapeutic treatment is described that leads to improved mood and immune status. The immune status is assessed based on total number of lymphocytes of T-lymphocytes (CD3), T-helpers (CD4), cytotoxic killers (CD8) and natural killers (CD16).

### SUMMARY OF INVENTION

The method and device outlined in this document of the present disclosure enable the enhancement of the neural activity associated with the processing of various perceptual, psychological and/or cognitive events or tasks. The method and device taught in the present disclosure further relate to the enhancement of the neural processing of various types of sensory information by placing the brain in a state best suited for processing said information or task. The method and device can be used to treat and alleviate depression.

In the present disclosure, the terms “task” and “perceptual, psychological and/or cognitive event” are to be understood to be synonymous. The task elicits, upon provision to, and perception by, a user, the neural activity associated with the processing of the task. An aim or goal is to be understood to be a behavioural aspect of the task. The aim or goal can be compared with an outcome of a behavioural response of the user to the task.

The task may be a passive task, such as when providing to a user a perceptual event, e.g., a visual, auditory, somatosensory, tactile, olfactory, gustatory, or vestibular event that does not include an aim or a goal. An example of a passive task would be one or more user's being played a sequence of auditory sounds over headphones, which requires the user to passively perceive the task without requiring an active response or reaction from the user. The passive task may not require the user to perform any voluntary actions in response to the stimulus, but merely to attend to the stimuli being presented.

The task may be an active task, such as when providing to the user an event that is perceptual and cognitive, e.g., the afore-mentioned visual, auditory, somatosensory, olfactory, gustatory, or vestibular event that includes an aim or a goal. An example of an active task would be in which the user(s) are instructed by a voice played over headphones to imagine a scene or scenario (e.g., the expression on a known person's face when this person is happy), which requires the user to actively follow the instructions given to them. Alternatively, the user may hear a sequence of different words presented auditorily via headphones and be instructed to classify each word (via tapping on a button on a smartphone display) as an emotional or non-emotional word, which requires the user to actively follow instructions given to them and provide a response based on this active instruction. The active task may require the active participation of the user. The active task may require the user to perform a voluntary action that may or may not produce a measurable response from the user.

A task may comprise one or more instructions. The one or more instructions ask or require the user to perform one or more actions on subsequent content presented during the task. The task further comprises the content upon which the users is asked or required to perform the one or more actions. The task may further comprise a response by the user. The response may be based on the one or more instructions, one or more actions, and/or the content.

The processing of any task or perceptual, psychological and/or cognitive event by an individual results in a “pattern” of neural activity. This pattern of neural activity is understood to refer to the activity of distributed neural circuits located across different regions of the brain, i.e., the interconnected network of neurons, the neural activity of which is elicited by the task. The pattern of neural activity may refer to similarities in the neural activity across the distributed neural circuits or across the brain, as opposed to the individual neurons that fire. The pattern of the neural activity is further to be understood to mean the time course of the neural activity of which is elicited by the task.

In the present disclosure, the term “sensory stimulus” is to be understood to mean a stimulus that is provided to the user and perceived by the senses or the sensory system of the user. The sensory stimulus includes visual, auditory, somatosensory, olfactory, gustatory, and vestibular stimuli.

The present disclosure provides a device and method. The method comprises two interrelated steps that are administered or provided to the user together. The two steps may be provided at least partially simultaneously or consecutively within a predetermined window of time. In the present disclosure, a single presentation of these two interrelated steps is called a “stimulus-task-cycle”.

In the present disclosure, the term “session” refers to single period of time in which the user is presented a series of stimulus-task-cycles. In each session the user may first be given instructions, indicating the requirements of their current task. The users may then be shown one or more stimulus-task-cycles in which the users perform the instructed task in response to sensory input or stimulus presented to them during each stimulus-task-cycle.

The invention is defined by the appended claims.

A computing system for treatment of a neural disorder of a user according to the present disclosure comprises one or more processors, a memory, and one or more output devices. The computing system further comprises an oscillator module stored in the memory and adapted to run on the one or more processors, thereby interacting with the output device to produce rhythmic sensory stimuli, perceivable by senses of the user, at at least one predetermined frequency to induce oscillations in a brain of the user and thereby to enhance neural activity of a neural circuit associated with the processing of a task. The computing system further comprises a content module stored in the memory and adapted to run on the one or more processors, thereby interacting with the output device to produce content to engage the user in the task.

The one or more output devices may comprise at least one of an auditory device, a visual device, or a tactile device. The one or more output devices may further comprise a nasal implant, such as electrodes placed in the nose of the user, e.g., in the sinus cavities, a cochlear implant, a vestibular implant, and a tactile stimulator.

The rhythmic sensory stimuli may comprise at least one of visual stimuli, auditory stimuli, somatosensory stimuli, olfactory stimuli, gustatory stimuli, and vestibular stimuli.

The rhythmic sensory stimuli may additionally comprise non-rhythmic sensory stimuli.

The content may comprise at least one of a visual task, an auditory task, a somatosensory task, an olfactory task, a gustatory task, and a vestibular task.

The computer system may further comprise measuring devices to measure activity of the brain.

A method for treatment of a neural disorder in a user according to the present disclosure comprises providing rhythmic sensory stimuli, perceivable by senses of the user, at at least one predetermined frequency to induce oscillations in a brain of the user and thereby to enhance neural activity of a neural circuit associated with the processing of a task.

The method further comprises engaging the user in the task.

The providing of the rhythmic sensory stimuli and the engaging in the task may occur within a predetermined window of time.

The providing of the rhythmic sensory stimuli and the engaging in the task may occur at distinct times or at least partially simultaneously.

The at least one predetermined frequency may be selected from the range of between 0 Hz and up to 300 Hz.

The rhythmic sensory stimuli may comprise at least one of visual stimuli, auditory stimuli, somatosensory stimuli, olfactory stimuli, gustatory stimuli, and vestibular stimuli.

The rhythmic sensory stimuli may in addition comprise non-rhythmic sensory stimuli.

The engaging in the task may comprise providing at least one of a visual task, an auditory task, a somatosensory task, an olfactory task, a gustatory task, and a vestibular task.

The engaging in the task may comprise providing task-related instructions to the user.

The providing of the rhythmic sensory stimuli may be carried out using one or more output devices.

The providing of the rhythmic sensory stimuli may comprise presenting a sensory stimulus by means of one or more output devices, wherein a first parameter of the presentation is changed at a first one of the at least one predetermined frequency, and a second parameter of the presentation is changed at a second one of the at least one predetermined frequency.

The engaging in the task may comprise at least one of rating a displayed picture, memorisation of one or more facts, measuring a reaction time, making a decision, actively attending to a stimulus, or a breathing exercise.

The at least one predetermined frequency may lie in the frequency range of alpha oscillations, beta oscillations, delta oscillations, gamma oscillations or theta oscillations.

The method may further comprise measurement of neural activity.

The variation of the at least one predetermined frequency may be dependent on the measured neural activity.

The method may further comprise repeating the method of the present disclosure.

The repetition may take place over the course of one or more days.

A use of the method according to the present disclosure for the treatment of at least one of depression or anxiety or for the enhancement of immunity is disclosed. The use of the method according to the present disclosure is further disclosed for the treatment of at least one of disruptive mood dysregulation disorder, major depressive disorder, dysthymia, premenstrual dysphoric disorder, separation anxiety disorder, social phobia, agoraphobia, generalised anxiety disorder, acute stress disorder adjustment disorders, illness anxiety disorder, bipolar I and II disorders, cyclothymic disorder, substance/medication-induced depressive, anxiety, or bipolar disorder, or similar.

One step of the method provides the user with rhythmical sensory stimuli to induce neural oscillations associated with the brain states. A further step of the method comprises exposing the user to a task to elicit neural activity associated with the processing of the task or perceptual, psychological and/or cognitive event. The exposing of the user to the task can at least partially occur simultaneously with the providing of rhythmical sensory stimuli to the user. The method induces both neural oscillations and task-related neural activity in a user's brain.

The method according to the present invention modifies the preceding and/or concurrent neural activity associated with the processing of the task via the presentation of the rhythmic sensory stimuli by an inducer module or oscillator module. Relative to processing the task alone, the additional presentation of the rhythmic sensory stimuli enhances the neural response to the task and/or reduces the undesired alteration in the present neural activity and/or improves the processing of the task. The frequency or frequencies that are induced by the rhythmic sensory stimuli, when targeting the intervention towards a selected indication, are therefore based on which ones or types of the neural oscillations (or set of neural oscillations) will best enhance the processing of the task (and thus reinforce the activity of the desired neural circuit).

An extensive body of research demonstrates that the neural oscillations exhibit strong frequency-specific correlations with perceptual and cognitive processing. For example, gamma oscillations have been implicated in feature binding, memory processes, and crucially, feedforward processing. Additionally, gamma oscillations have been shown to be tightly linked (i.e., ‘coupled’) with the timing of theta oscillations. Together, coupled theta-gamma oscillations are believed to facilitate the transfer of information across distant brain areas and support higher-level perceptual and cognitive processing. Thus, stimulating early visual processing areas at gamma and/or theta frequencies should facilitate enhanced propagation of simple stimulus features through the visual hierarchy. This mechanism of strengthened feedforward processing constitutes the enhanced processing of visual stimuli, regardless of their complexity.

The role of the rhythmic stimuli in this context is to boost the initial processing, propagating the flow of information to the relevant regions, i.e., neural circuits of the brain. Specifically, the viewing of gamma frequency rhythmic stimuli (i.e., auditory and visual stimuli that are modulated so that their intensity fluctuates with a sinusoidal, square, triangle, sawtooth, or other waveform at a frequency between 35 and 70 Hz) results in a modification of the equivalent gamma frequency oscillation band in the brain.

The induction of the gamma oscillations has also been linked to enhanced neuroplasticity, with the gamma oscillations thought to be important in the establishment of long-term changes in response to increased or decreased activity of a circuit. Neuroplasticity is a mechanism in the brain through which the neurons adapt the strength of their connections depending on their activity, with the neurons that frequently fire together at the same time growing stronger connections, while those neutrons that are not engaged together become less likely to activate together.

The device and method can utilize the gamma oscillations and be combined with various tasks to ensure that the enhanced neural activity of the task-related neural circuits can result in long-term changes in the neural circuit. In this way, an underactive neural circuit can be strengthened and return to normal activity levels by the targeted engagement of the neural circuit via the presentation of a carefully chosen task to selectively engage the underactive circuit in question. The induced gamma oscillations both enhance the strength of the task-related circuit activation and increase the neural plasticity to promote a long-term change in the neural circuit.

The literature also indicates that the combination of induced oscillatory activity and stimulus can act in the opposite direction and suppress the processing of information. Alpha oscillations, for instance, have been well evidenced as operating an inhibitive functional role in visual information processing. This slower rhythm plays a key role in suppressing irrelevant environmental information, which functions to protect memory formation. The providing of an alpha oscillation can be useful in users wishing to enhance their memory processing, or where the alpha oscillations act to suppress irrelevant or unwanted stimuli, helping the user focus attention on the relevant stimulus in question.

The device and method seek to shift the brain state prior to the task or stimulus into the optimal state for deriving the required neural changes. In this way, every trial of the intervention according to the present device and method contributes to the desired neural changes and exerts an optimal effect on the targeted neural circuits for future processing of the task or perceptual and/or psychological and/or cognitive event processed by the targeted neural circuits. This makes a target stimulus-based intervention a pragmatic and practical alternative, with modification of afflicted or targeted neural circuits achievable with reasonable durations across a reasonable window of time, rather than the prohibitively frequent repetitions that would be required from non-optimal engagement of the afflicted or targeted neural circuits alone.

The method and device of this document act to enhance the successful encoding of various types of stimuli. This enables exerting a stronger and long-lasting change on the circuits involved in processing the task. For instance, if a user has a specific deficit in their ability to process faces, the gamma oscillation in conjunction with face stimuli could act to enhance such processing via strengthening the flow of facial information from the simple contours of the eyes to the recognizable holistic representation of a face. This bottom-up approach, while stimulating the early visual cortex, has consequences on higher-level processing regions. Here, the more abstract brain regions the user wishes to stimulate is reached by the selecting of an appropriate task that selectively engages the circuits of interest.

The present disclosure is further directed to a non-pharmacological approach for managing chronic pain. The present disclosure in particular further relates to a combination treatment of pain comprising both visual exposure of a user to green light and the induction of alpha waves in the user's brain by means of visual and/or auditory sensory stimuli. The method thus enables a synergistic analgesic effect by acting simultaneously on distinct physiological pathways which may interact with one another and potentiate the analgesic effect. The disclosure further relates to a system and a method for treatment of pain that enables increased endogenous analgesia and decreased pain salience.

The method may further comprise the step of exposing the user to green light The green light results in nociceptive (i.e., pain-sensation-inhibiting) effects and increases alpha band activity.

The providing of the rhythmic sensory stimuli may comprise the exposing of the user to the green light. The visual rhythmic sensory stimuli may comprise the green light.

The green light may have a wavelength of between 495 nm and 570 nm, preferably between 505 nm and 545 nm, and most preferably of approximately 525 nm.

The green light may have a luminance of between 0 and 20 lux, preferably between 5 and 15 lux, most preferably of approximately 10 lux.

The green light may be provided by means of at least one light emitting source.

The at least one light emitting source may be a display.

The method may further comprise guiding the green light to at least one eye of the user.

The green light may be provided as at least one of green flicker, a green tint, and a green frame.

The green flicker may comprise at least one of light flashes, moving objects or patterns.

The at least one rhythmic sensory stimulus may oscillate with a frequency lying in a frequency range between 0 and 200 Hz, preferably between 1 and 20 Hz, more preferably of between 6 and 14 Hz.

The at least one rhythmic sensory stimuli may comprise at least one of an auditory stimulus, a visual stimulus, a somatosensory stimulus, a tactile stimulus, an olfactory stimulus, a gustatory stimulus, or a vestibular stimulus.

The at least one rhythmic sensory stimulus may comprise at least one of sound or music.

The method may further comprise initiating the treatment based on body parameters of the user.

The method may further comprise the step of sensing the body parameters of the user.

The body parameters may relate to at least one of subjective report, heart rate, heart rate variability, speech, electrodermal activity, movement, respiratory rate, and blood pressure.

A computing system for treatment of a health disorder is disclosed. The computing system comprises one or more processors; a memory; one or more output devices; an oscillator module, stored in the memory and adapted to run on the one or more processors to produce rhythmic sensory stimuli by means of at least one of the one or more output devices; a green light module stored in the memory and adapted to run on the one or more processors to produce green light by means of at least one of the one or more output devices.

The one or more output devices may be configured to provide at least one of a visual stimulus, a somatosensory stimulus, an olfactory stimulus, a gustatory stimulus, or a vestibular stimulus.

The one or more output devices may comprise at least one of a display, one or more LEDs, a wired or wireless interface for auditory signals, or a loudspeaker.

The computing system may comprise at least one of a mobile device or a wearable device.

A computer program product is disclosed. The computer program product, when executed on a processor, performs the steps of the method of the present disclosure.

The present disclosure relates to a use of the method of the present disclosure for relief of at least one of acute pain, chronic pain, migraine, mood disorders, major depressive disorder, anxiety disorder, bipolar disorder, substance-related disorders, addictive disorders, alcohol use disorder, substance use disorder, disorder due to use of cannabis or cannabinoids, disorder due to the use of nicotine, gambling disorder.

The present disclosure is further directed to a non-pharmacological approach for managing a circadian rhythm-related disorder.

A method of treatment of a circadian rhythm-related disorder of a user, the method comprising the steps of providing rhythmic sensory stimuli, perceivable by senses of the user, at at least one predetermined frequency to induce oscillations in a brain of the user and thereby to enhance neural activity of a neural circuit associated with the processing of a task. The method further comprises the step of engaging the user in the task, wherein the predetermined frequency and the task are chosen according to a time of the day at which the user conducts a session of the method.

The task may comprise visual content and/or audio content configured to induce a pre-defined mood in the user.

The visual content may comprise goal-oriented images, images directed to one or more of family, friends, holidays, leisure activities, and healthy lifestyle, when the time of the day is in the morning, or the visual content may comprise images that comprise scenes in ambient lighting and are one or more of non-food-related, calming, and sleep-inducing, when the time of the day is in the evening.

The audio content may comprise music, when the time of the day is in the morning, or the audio content may comprise calming instrumental music, when the time of the day is in the evening.

The task may further comprise one or more of a rating task, a savouring task, answering questions, a motivational task, when the time of the day is in the morning, or the task may further comprise one or more of performing a breathing exercise, keeping a motivation diary, performing a yoga exercise, when the time of the day is in the evening.

The task may further comprise time-restricted feeding and/or regular sleep habits.

The rhythmic sensory stimuli may be provided visually and/or transcranially.

The rhythmic sensory stimuli may comprise at least one of auditory rhythmic stimuli, visual rhythmic stimuli, or somatosensory stimuli.

The rhythmic sensory stimuli may have a frequency of 13 to 25 Hz, when the time of the day is in the morning, or the rhythmic sensory stimuli may have a frequency of 4 to 13 Hz, when the time of the day is in the evening.

The rhythmic sensory stimuli may comprise visual stimuli configured to stimulate ipRGCs in one or more eyes of the user, when the time of the day is in the morning, or the rhythmic sensory stimuli may comprise visual stimuli configured not to stimulate ipRGCs in one or more eyes of the user, when the time of the day is in the evening.

The visual stimuli may be non-rhythmic.

The visual stimuli may comprise light, having a wavelength of between 430 nm and 515 nm, preferably between 450 nm and 495 nm, or a colour temperature of between 5000 K and 27000 K, preferably between 6500 K and 9500 K, when the time of the day is in the morning.

The visual stimuli may have a brightness of 500 lux to 1000 lux at the one or more eyes or an ear channel of the user, when the time of the day is in the morning.

The visual stimuli may comprise light, having a wavelength equal to or greater than 500 nm, preferably between 500 nm and 565 nm, or a colour temperature of 1000 K to 3000 K, when the time of the day is in the evening.

The visual stimuli may have a brightness of between 10 lux and 1000 lux, preferably 10 lux and 100 lux when at the one or more eyes or the ear channel of the user, when the time of the day is in the evening.

The user may initiate or continue the session based on body parameters of the user.

The method may comprise detecting the body parameters.

The body parameters may relate to at least one of chronotype, body temperature, sleep patterns, heart rate, heart rate variability, skin conductance, breathing patterns, blood pressure, and brain waves.

The circadian rhythm-related disorder may comprise night eating syndrome, obesity, jet lag, shift work disorder, binge eating disorder, food addiction, and metabolic syndrome.

A computing system for treatment of a circadian rhythm-related disorder of a user is disclosed. The computing system comprises one or more processors; a memory; one or more output devices; an oscillator module, configured to run on the one or more processors to produce rhythmic sensory stimuli by means of at least one of the one or more output devices, the rhythmic sensory stimuli being, perceivable by senses of the user, at at least one predetermined frequency to induce oscillations in a brain of the user and thereby to enhance neural activity of a neural circuit associated with the processing of a task. The computing system further comprises a content module configured to run on the one or more processors to provide, by means of at least one of the one or more output devices, the user with the task to engage the user in the task.

The task may comprise visual content and/or audio content configured to induce a pre-defined mood in the user.

The content module may comprise a behaviour module configured to run on the one or more processors to produce, by means of at least one of the one or more output devices, behavioural stimuli configured to induce behavioural patterns relating to feeding patterns.

The oscillator module may comprise a light module configured to be controlled by the one or more processors to produce, by means of at least one of the one or more output devices, visual stimuli configured to stimulate ipRGCs in one or more eyes of the user or visual stimuli configured not to stimulate ipRGCs in the one or more eyes of the user.

The one or more output devices may comprise at least one of an auditory device and a visual device.

The one or more output devices may comprise at least one of a display, an earbud, an audio jack, a loudspeaker, and a vibration motor.

The memory may store data relating at least one of the rhythmic sensory stimuli, the visual stimuli, the visual content, the audio content, and/or the behavioural stimuli.

The computing system further comprises at least one sensor configured to sense body parameters.

The body parameters may relate to at least one of chronotype, body temperature, sleep patterns, heart rate, heart rate variability, skin conductance, breathing patterns, blood pressure, and brain waves.

The computing system may comprise at least one of a smartphone or a VR headset.

A computer program product is disclosed, which when executed on a processor, performs the steps of the method of treatment of a circadian rhythm-related disorder of a user.

A use of the method of treatment of a circadian rhythm-related disorder of a user is disclosed for the treatment of Night Eating Syndrome, obesity, jet lag, shift work disorder, binge-eating disorder, food addiction, metabolic syndrome.

### DESCRIPTION OF THE FIGURES

FIG. 1 shows an aspect of a computing system according to the present disclosure.

FIG. 2 shows an aspect of a method according to the present disclosure.

FIG. 3 shows possible temporal profiles of presenting a sensory stimulus (inducer stimulus) during an inducer period and a task (content image) during a content period according to the present disclosure.

FIGS. 4A-4E show possible temporal and spatial profiles of presenting a sensory stimulus (inducer stimulus) and a task (content image) according to the present disclosure.

FIG. 5 shows possible stimulus-task-cycles or possible series of stimulus-task-cycles as well as instructions for passive and active tasks.

FIG. 6 shows possible images from the content module for use in a depression-focused implementation according to the present disclosure.

FIG. 7 shows results of measured effect size of the reported mood and anxiety changes after a single 10-minute session with an active (image savouring) or passive (image viewing) task provided by the content module according to the present disclosure.

FIG. 8 shows results of measured effect size of reported mood and anxiety changes after a single 10-minute session of conducting of the method of the present disclosure, in which the content module was optimised to target positive mood circuits or anxiety circuits.

FIG. 9 shows results of measured effect size of reported mood and anxiety changes after a single 10-minute session of conducting the method of the present disclosure, in which the content module provided a task optimised to target positive mood circuits or a control task.

FIG. 10 shows differences in the oscillation synchronization recorded in a user's brain in response to an effective versus an ineffective inducer.

FIG. 11 shows an increase in power at the targeted frequency in the brain for a variety of different inducers targeting different frequency bands.

FIG. 12 shows EEG data demonstrating that the presence of the inducer enhances the processing of emotional stimuli as evidence by a faster P3 peak in the ERP signal.

FIG. 13 illustrates a method according to the present disclosure.

FIG. 14 shows results of depressive symptom reduction after 6 and 2 weeks of 4-7 weekly sessions of the method of the present disclosure.

### DETAILED DESCRIPTION OF THE INVENTION

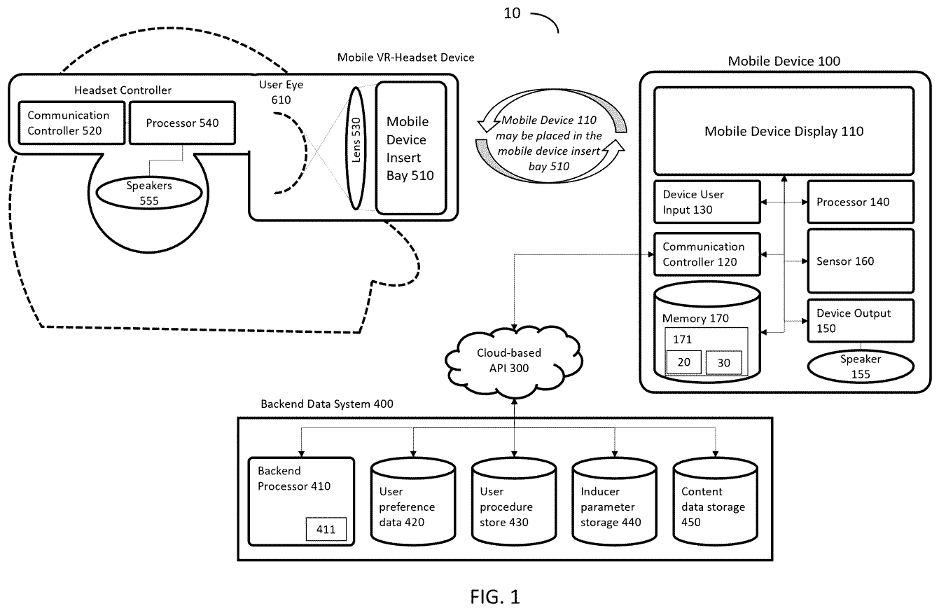

FIG. 1 shows an overview of the device 10 according to one aspect of the present disclosure. In aspect, the device 10 may be a computing system 10 for the treatment of a mood-related disorder, such as depression. The computing system 10 may comprise a computing device 100. The computing device 100 may be a mobile device, such as but not limited to a smartphone, or could be part of a cloud environment.

The computing device 100 comprises a display 110. The computing device 100 further comprises one or more communication controllers 120 comprising, for example, a Bluetooth controller, a Wi-Fi controller, and a cellular data controller. The computing device 100 comprises one or more user input devices 130 comprising a touchscreen, a microphone, and a camera. Other user input devices, such as a keyboard or a joystick, may be connected to the computing device. The computing device 100 further comprises one or more processors 140 including an audio processor, a graphics processor (e.g., a GPU), and a system processor (e.g., a CPU). The computing device 100 may further comprise one or more device outputs 150 comprising a haptic module, a vibrational module, audio jack and/or a speaker 155. The computing device 100 may further comprise one or more device sensors 160 comprising an accelerometer, a gyroscope, and a magnetometer.

The computing device 100 further comprises a memory or file storage 170. One or more programs 171 may be stored on the memory 170. The one or more processors 140 are configured to execute the one or more programs 171 stored on the memory 170. The one or more programs 171 may be programmed, upon execution of the one or more programs 171 by the one or more processors 140, to treat a user suffering from a mood-related disorder, as will be explained later.

The one or more programs 171 are configured to run on the computing device 100 so that the user can interact with the one or more programs 171 via the various user input devices 130 while viewing outputs presented on the display 110 and/or perceiving output presented by means of the one or more device outputs 150. In one aspect, the visual output may be generated by the graphics processor, auditory output may be produced by the audio processor via either the in-built speaker 155, by another speaker device connected via the audio jack, or by another speaker device connected via one of the one or more communication controllers 120, for example, via the Bluetooth controller for an external Bluetooth speaker.

The one or more programs 171 are configured such that the user can input user preferences 420. For instance, the user preferences 420 may be input via the device user inputs 130 by responding to questions presented via the display 110 and/or the device user inputs 130. In one aspect, this data is transmitted via the one or more communication controllers 120 to an API 300 which handles the exchange of data between the computing device 100 and a backend data system 400. The API 300 may be a cloud-based API 300.

The backend data system 400 comprises a backend processor 410, which executes various backend programs 411 in response to requests transmitted via the API 300 by the one or more programs 171 stored in the memory 170. The backend data system 400 enables the storage of user preferences 420, user procedures 430, inducer parameters 440 and content data 450 such that required data can be requested by the computing device 100 via the API 300 and transmitted to the computing device 100. Additionally, the backend programs 411 can perform operations within the backend processor 410 on the uploaded user preference data 420 to determine the best user procedure 430, inducer parameters 440 or content data 450 to return to the computing device 100 for presentation to the user.

Once the required data has been transmitted from the backend data system 400 to the computing device 100 the data is stored within the memory 170. When the one or more programs 171 stored in the memory 170 are operating, the user can initiate a session according to the present disclosure via selecting, by means of the display 110 and/or the one or more device user inputs 130, an option, e.g., displayed on the display 110 or transmitted by the one or more device outputs 150. The one or more programs 171 stored in the memory 170 are then executed to present information to the user.

The computing system 10 may further comprise a VR-headset 500. These VR-headset may include device-linked VR headsets (e.g., PlayStation VR, Oculus Rift/Rift S, HTC Vive/Vice Pro), dedicated stand-alone VR headsets (e.g. Oculus Go), or mobile-device based R headsets (e.g. Samsung Gear VR, Google Daydream view, Google Cardboard, VR Shark X4 VR, Pimax VR Glasses, SDYAYFGE 3D VR Glasses, Zeiss Virtual Reality ONE Headset) such as shown in 500. Instead of a VR-headset, the device may compromise an AR-headset (augmented reality) or mixed-reality headset, in which the displayed stimuli are optically overlaid on the external world or in which the external world is recorded via a camera to create a digital overlay.

The information presented to the user may comprise instructions for connecting the computing device 100 with the VR-headset 500. The VR-headset 500 may comprise a controller or gaming controller for manual input and/or manual control. The VR-headset 500 may comprise eye-tracking sensors. In one aspect, the connecting of the computing device 100 and the VR-headset 500 comprises interfacing the computing device 100 and the VR-headset 500 via the one or more communication controllers 120 of the computing device 100 and one or more communication controllers 520 of the VR-headset 500. The one or more communication controllers 520 may comprise a Bluetooth communication controller. For example, linking the computing device Bluetooth communication controller 120 to the VR-headset Bluetooth communication controller 520 enables audio generated on the computing device 100 by the audio processor to be transmitted to the VR-headset 500. The audio received by the VR-headset 500 may be processed by one or more onboard processors 540 of the VR-headset 500. The one or more processors 540 may comprise an audio processor. The audio may be played by inbuilt speakers 555 of the VR-headset 500. In further aspect, the user may connect their own headphones to the device audio jack. In another aspect, the user can rely upon the output from the inbuilt speaker 155 of the computing device 100.

The computing device 100 may be placed within a computing device insertion bay 510 of the VR-headset 500. The computing device insertion bay 510 provides a mechanical connection that restrains the computing device 100 within the VR-headset 500 and ensures the display 110 of the computing device 100 remains securely in place at an ideal physical distance from a lens 530 of the VR-headset 500. The effect of the lens 530 is that an image, e.g., a visual stimulus or a visually presented perceptual, psychological, and/or cognitive event, displayed on the display 110 appears to be both larger and further away to the user eye 610 than the actual physical distance of the display 110 in the VR headset. This ensures that the user can comfortably view the image displayed on the display 110. The lens 530 optically transforms the image of the display 110.

Upon securing the computing device within computing device insertion bay 510 of the VR-headset 500 and attaching the VR-headset 500 to the user's head, the user can begin a session with the device 10. In one aspect, the user presses a mechanical button arranged on the VR-headset 500, e.g., with a finger. The mechanical button generates in this aspect an input to the computing device 100 and initiates the session of conducting a method according to the present disclosure.

Alternatively, the session may be run directly on the display 110, without use of the VR-headset 500. In this case, the user may interact with the visual signals, e.g., stimuli or a task, via the display 110. The user may further interact with the auditory signals, e.g., auditory stimuli or an auditory task, via a device connected to the audio output jack of the computing device 100 or via the in-built speaker 155 of the computing device 100.

During the session, the one or more programs 171, e.g., the inducer module 20 or content module 30, may present to the user sensory stimuli. The sensory stimuli presented by the inducer module 20 are generated based on inducer parameters, which are stored in the inducer parameter storage 440 and acquired by the inducer module 30 via the API 300. The inducer parameters 440 may be used in mathematical equations. Based on the inducer parameters 440, the mathematical equations are evaluated to generate desired visual patterns to be displayed on the display 110. The sensory stimuli are generated, by one or more of the processers 140 of the computing device 100 based on the inducer parameters 440 acquired via the API 300.

For example, a visual inducer stimulus might be produced based on ones of the inducer parameters associated with a mathematical equation that defines a 2D matrix that is modulated by a sinusoid function to create a grating pattern (e.g., an alternating pattern of white and black bars). The modulation may be done along one dimension of the 2D matrix, in which case the inducer parameters comprise an amplitude (representing discrete black or white or representing any shade in between and including black and white), a spatial frequency (representing the scale of the grating), and a phase (representing a spatial offset). The modulation may be done along two dimensions of the 2D matrix, in which case the inducer parameters comprise one amplitude, two spatial frequencies, and two phases.

The one or more programs 171, e.g., the content module 30, may further display to the user content images generated based on content data, which are stored in the content data storage 450 and acquired by the content module 30 via the API 300. In one aspect, the content data 450 comprise content images that can be displayed via the display 110.

Additionally, an auditory signal may be acquired via the API 300 from the inducer parameter storage 440 of the backend data system 400 or generated via the one or more processors 140. In one aspect, the auditory inducer stimulus might be generated based on ones of the inducer parameters associated with a mathematical equation that describes an oscillating tone. The inducer parameters may in this case comprise an amplitude (representing the auditory volume of the tone), a temporal frequency (representing the pitch of the tone), and a phase (representing a temporal offset). Modulation of the auditory inducer can be achieved via frequency and/or amplitude modulation. The modulation may comprise one or more target frequencies. The modulation may be directed at the same or different target frequencies.

The auditory signal may be presented via various auditory output methods at the same time as the presentation of the visual pattern via the display 110. Both auditory and visual stimuli are generated based on a clock within the one or more processors 140, e.g., the system processor, of the computing device 143 to ensure the auditory and visual stimuli are presented in synchrony.

In one aspect, following approximately 5-10 seconds of presentation of a combination of the auditory and visual inducer stimulus, the content image is presented for 5-10 seconds. However, the auditory and visual inducer stimulus as well as the content image may be presented for shorter or longer durations. The content image may comprise a picture or photograph of an animal, human, landscape, or architectural image that is acquired from the content image storage 450 via the API 300 and stored within memory 170 until required. When presented the content image is processed via the graphics processor 142 so that the content image can be shown either directly on the display 110 or indirectly via the lens 530 of the VR-headset 500 when the computing device 100 has been arranged within the VR device insert bay 510. During the presentation of the content image the user may additionally hear a continuation of the auditory stimulus presented by the inducer module 20 or an auditory content signal, acquired via the API 300 from the content data storage 450. After the presentation of the content image, the one or more programs 171 may then request a user response from the user via the one or more device user input 130 depending on the specific task. This user response may include accessing the touchscreen, the gyroscope, the accelerometer, the camera and/or the microphone. Once a user response has been processed, the processed user response can then be uploaded to the backend data system 400 via the API 300 and used for future sessions of the device 10.

The computing system 10 may further comprise one or more measuring devices to measure activity of the brain. The one or more measuring devices comprise an EEG device (electroencephalography device). The one or more measuring devices may measure the activity of the brain based on physiological parameters such as heart rate variability or skin conductance. The one or more measuring devices may further comprise devices for functional neuroimaging such implementing techniques such as functional magnetic resonance imaging (fMRI), functional near-infrared spectroscopy (fNIRS), magnetoencephalography (MEG), functional ultrasound imaging (fUS).

The computing system 10 comprises a coupled relationship between an oscillator module or inducer module 20 and a content module 30. The one or more programs 171 may comprise the oscillator module 20 and the content module 30. The one or more programs 171 may comprise further modules 40 (see below). The oscillator module 20 and the content module 30 may be accessed by the one or more processors 140, e.g., by the system processor. The oscillator module 20 and the content module 30 may be executed by the one or more processors 140, e.g., by the system processor. The oscillator module or inducer module 20 generates a rhythmic sensory stimulus, presented as a visual, auditory, somatosensory, tactile, olfactory, gustatory, or vestibular stimulus to the user. The sensory stimulus occurs before or partially overlapping with a task or perceptual, psychological, and/or cognitive event generated by the content module 30. The role of the oscillator module 20 is to influence the preceding and ongoing brain state for when the user is exposed to the task generated by the content module. In some respects, the stimuli generated by the oscillator module 20 will be presented prior to the presentation of the stimuli generated by the content module 30. In other instances, there is either a partial or complete overlap such that the stimuli generated by the content module 30 are presented concurrently with rhythmic stimuli generated by the oscillator module 20.

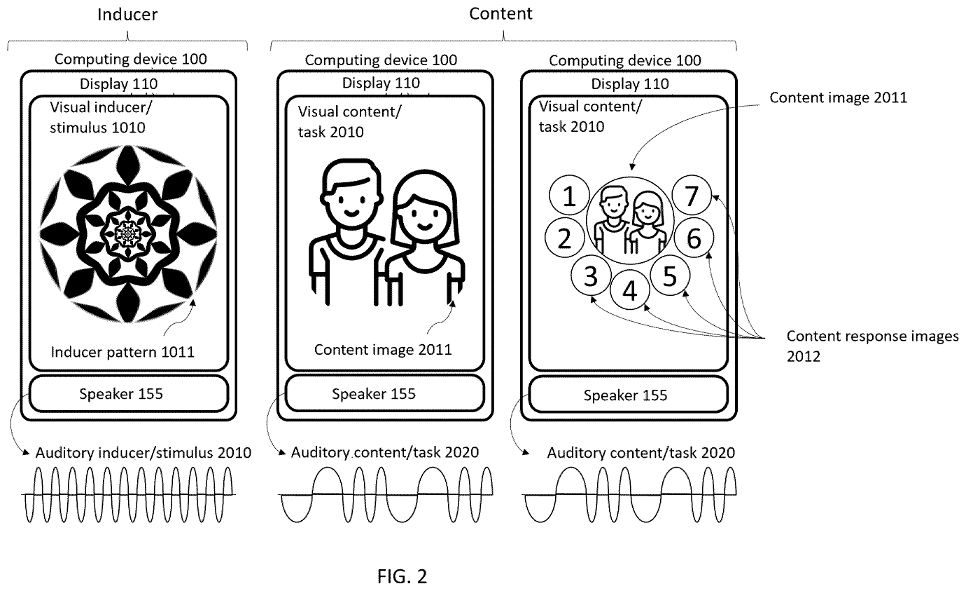

FIG. 2 shows an aspect of a session according to the present disclosure for treating depression. The session comprises at least one inducer period and at least one content period. The at least one inducer period and the at least one content period occur within a predetermined window of time, referred to as stimulus-task-cycle. This ensures that that the induced neural activity and the processing of the task are not independent. In other words, the neuronal response to the sensory stimulus and the behavioural response to the task are not independent. The session may comprise one or more stimulus-task-cycles. In one aspect, the inducer period and the content period may occur at distinct times within the stimulus-task-cycle. In another aspect, the inducer period and the content period may at least partially temporally overlap, i.e., comprise common times within the stimulus-task-cycle. In one aspect, the stimulus-task-cycle has a constant duration during the session with a user. The duration of the stimulus-task-cycle may be in range of 1 to 100 seconds. However, durations of the stimulus-task-cycle such as 1-10 minutes are conceivable. In another aspect, the stimulus-task-cycle has a variable duration during the session with a user.

During the inducer period, the sensory stimuli are generated and presented to the user. The generated sensory stimuli may further include one or more of a visual, auditory, somatosensory, tactile, olfactory, gustatory, or vestibular stimulus.

For instance, examples of the visual stimuli 1010 comprise patterns 1011 which may be generated by the one or more processors 140 of the computing device 100, e.g., by the graphics processor, from parameters accessed via the API 300 and stored in the inducer parameter storage 440. The generated visual stimuli 1010 may be presented to the user, e.g., shown on the display 110.

Moreover, the auditory stimuli 1020 may be generated by means of the one or more processors 140, e.g., the computing device audio processor. The auditory stimuli 1020 may be presented to the user via the speakers 155 of the computing device 100.

During the content period, content, i.e., a task or perceptual and/or psychological and/or cognitive event is presented to the user. The task (or perceptual and/or psychological and/or cognitive event) includes a visual, auditory, somatosensory, tactile, olfactory, gustatory, or vestibular task.

For instance, the visual content 2010, i.e., a visual task, comprising a content image 2011 that has previously been downloaded via the API 300 from the content data storage 450, is shown on the display 110. For instance, auditory content 2020, i.e., an auditory task, acquired from the image content data storage 450 and possibly modified by the one or more processors 140, e.g., the auditory processor, is presented via the speakers 155 of the computing device.

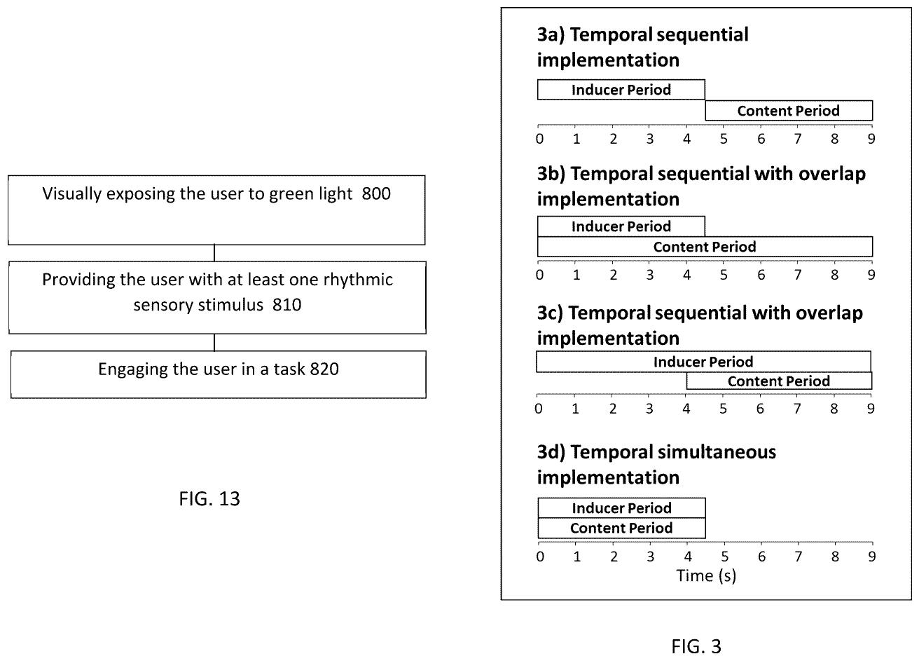

The inducer period and the content period may occur at distinct times or distinct time periods. The distinct times or distinct period do not have any temporal overlap, i.e., do not have any point in time in common. In another aspect, the inducer period and the content period may temporally overlap (see, e.g., the description of FIG. 3 below).

Depending on the implementation, the user may be required or asked to make a response to the presented visual content, i.e., the visual task or perceptual and/or psychological and/or cognitive event. In this aspect, content response images 2012 are presented, and the one or more device user inputs 130 and the one or more device sensors 160 are used to register a selection. For example, the gyroscope sensor may be used to make the selection by tilting the computing device 100. Alternatively, the selection may be registered directly on the display 110 via a touchscreen input.

As described below, the session of conducting the method according to the present disclosure may comprise one or more further periods in addition to the inducer period and the content period. During the one or more further periods, the user is presented with further ones of the stimuli, tasks, or events by means of the display 110 or by means of the one or more device outputs 150. The further ones of the stimuli, tasks, or events may differ from the rhythmic sensory stimuli provided to the user during the inducer period or from the content presented to the user during the content period. The one or more further periods may or may not overlap with the inducer period and the content period.

FIGS. 3A-3D shows some aspects and temporal profiles of the inducer period and the content period. The present disclosure is not, however, limited to the depicted temporal profiles and other temporal profiles may be chosen. The sensory stimulus and the task (or perceptual and/or psychological and/or cognitive event) are presented during the inducer period and the content period, respectively. The sensory stimulus and the task may be presented in different temporal configurations as can be seen in FIGS. 3A-3D. Moreover, the temporal profiles shown in FIGS. 3A-3D may comprise the further periods (mentioned above and not shown) during which the user is presented with the further ones of the stimuli, tasks, or events.

The temporal configurations include a temporal sequence (see FIG. 3A), in which case first the sensory stimulus is presented the inducer module 20 and the task is presented after the presentation of the sensory stimulus has finished. In other words, there is no temporal overlap of the inducer period and the content period. The temporal configurations further include a temporal sequence with partial overlap (see FIG. 3B), in which the task and the sensory stimulus are initially presented simultaneously, and the presentation of the task lasts longer than the presentation of the sensory stimulus. In other words, there is a temporal overlap of the of the inducer period and the content period, and the content period extends beyond the inducer period. The temporal configurations further include a temporal sequence with partial overlap (see FIG. 3C), in which the sensory stimulus is initially presented alone for a predetermined period, followed by a period during which the sensory stimulus and the task are presented simultaneously. The presentation of the sensory stimulus begins earlier and ends at the same moment as the presentation of the task. In other words, there is a temporal overlap of the inducer period and the content period, and the content period begins after the inducer period. The temporal configurations may further include a temporally simultaneous presentation of the sensory stimulus and task, in which case both the sensory stimulus and the task occur at the same time and for the same duration. In a further aspect, which is not shown, the temporal configurations may include a temporal sequence in which the inducer period begins before the content period, followed by a period during which the inducer period and the content period temporally overlap, and the inducer period ends before the content period ends. In other words, the sensory stimulus and the task are presented on their own at the beginning of the temporal sequence and at the end of the temporal sequence, respectively, with a period overlap between the beginning and the end. It will be understood that the further periods (mentioned above), during which the user may be present with the further ones of the stimulus, task, or event, may occur in parallel and/or serially with the inducer period and the content period.

When the inducer period and the content period temporally overlap, the sensory stimuli from the inducer module or oscillation module 20 and the task from the content module 30 are presented to the user simultaneously for the duration of the temporal overlap. In one aspect, the simultaneous presentation of the sensory stimulus and the task may occur by means of distinct sensory modalities. In another aspect, the simultaneous presentation of the sensory stimulus and the task may occur by means of the same sensory modality. For instance, in the case of distinct sensory modalities, the sensory stimulus may be a visual stimulus, and the task or perceptual, psychological, and/or cognitive event may be an auditory task or perceptual, psychological, and/or cognitive event. For instance, in the case of the same sensory modality, the sensory stimulus may be a visual stimulus, and the task or perceptual, psychological, and/or cognitive event may be a visual task or perceptual, psychological, and/or cognitive event. It will be understood that the further ones of the stimulus, task, or event (mentioned above) may be presented to the user by means of the same sensory modality or a different one of the sensory modality.

In an aspect, in which the sensory stimulus and the task are presented by means of the same sensory modality, and in which the inducer period and the content period temporally overlap, the sensory stimulus and the task are presented to the user in a manner that allows for their simultaneous presentation to the user.

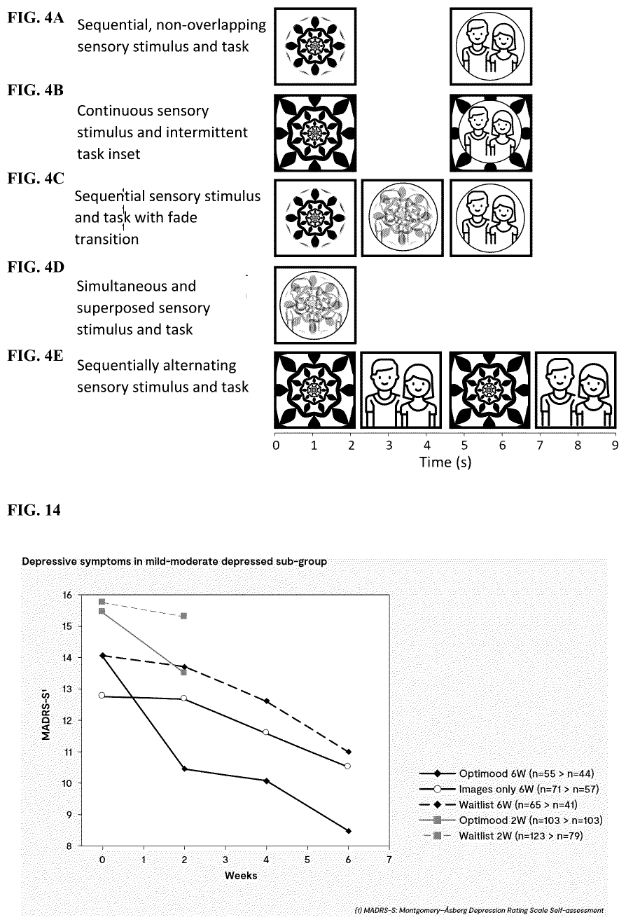

FIGS. 4A-4E show possible temporal and spatial arrangements of presenting to the user a visual stimulus and a visual task.

FIG. 4A shows a visual stimulus, i.e., a stimulus image, and a visual task, i.e., a task image, presented in a sequential, non-overlapping manner.

FIG. 4B shows a visual stimulus, i.e., a stimulus image, presented continuously and a visual task, i.e., a task image, intermittently presented within an inset of the image.

FIG. 4C shows a visual stimulus, i.e., a stimulus image, and a visual task, i.e., a task image, presented to the user, which fade-transition into one another, i.e., one of the stimulus image and the task image fades in while the other of the stimulus image and the task image fades out. In other words, the stimulus image and the task image are visually superposed onto one another with the stimulus image and the task image being visible to varying degrees during the inducer period and the content period.

FIG. 4D shows a visual stimulus, i.e., a stimulus image, and a visual task, i.e., a task image, presented simultaneously and superposed onto one another such that both the stimulus image and the task image are visible.

FIG. 4E shows a sensory stimulus and a task presented alternatingly with no periods of simultaneous presentation, i.e., with no temporal overlap of the inducer period and the content period.

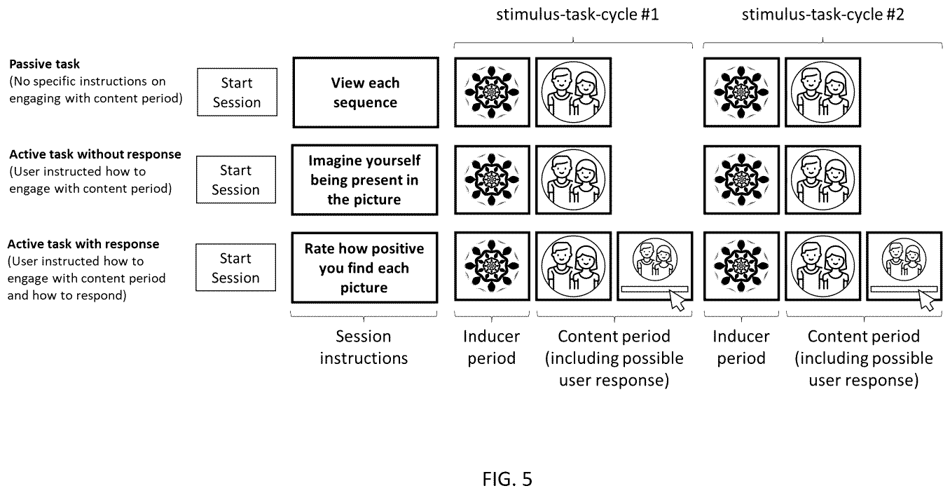

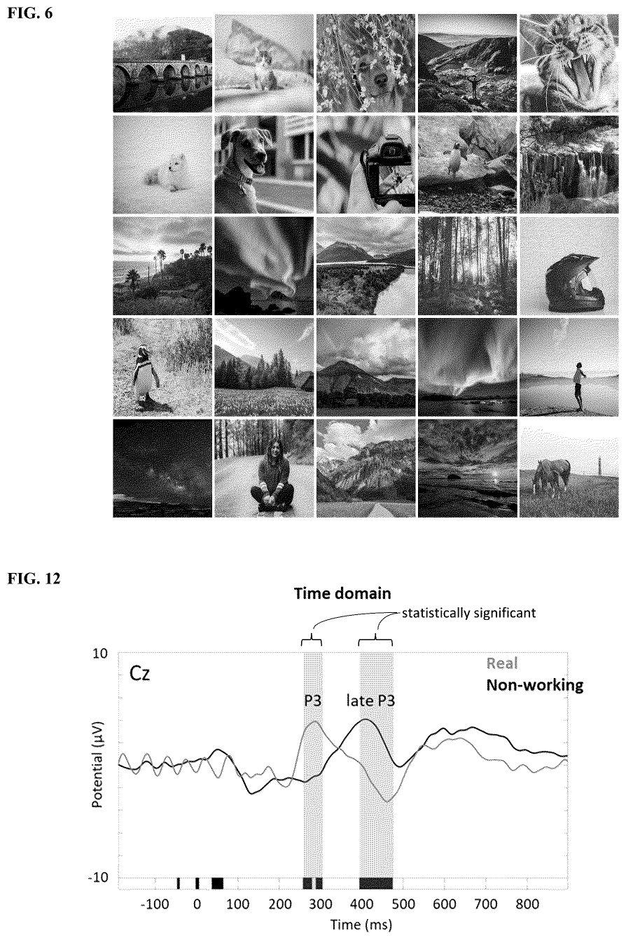

FIG. 6 shows a non-exhaustive list of possible task images presented during a visual task by the content module 30 to the user for use in a depression focused implementation of the present device and method with the goal engaging positive affective circuits in the brain of the user.

The oscillation presented by the oscillator module 20 is modality non-specific, as mentioned above. The specific manifestation or implementation can be in any singular modality (sensory modality), or it can be multisensory. Indeed, there is ample evidence that oscillations in different sensory modalities produce similar neural. The oscillator module 20 of this document enables the combination of distinct oscillations in different sensory domains (or modalities). These distinct oscillations are presented to the user by the one or more device outputs 150, i.e., by an appropriate one of the one or more device outputs 150 depending on the modality of the specific inducer stimulus, e.g., by the device display 110, the haptic module, the vibrational module, the audio jack and/or the speaker 155.

The oscillator module or inducer module 20 uses temporal parameters that define the presentation of the sensory stimuli provided by the oscillator module 20 to a user. The one or more processors 140 comprise the clock. As noted above, the oscillator module 20 is connected to the clock which provides timing. For instance, throughout the duration of a session of conducting the method of the present disclosure, all the temporal modulations of the sensory stimuli can be controlled by the inducer module or oscillator module 20 with respect to, and/or based on, timing signals from the clock.

Likewise, the content module 30 is connected to the clock. For instance, throughout the duration of a session of conducting the method of the present disclosure, the temporal modulations of the task or perceptual, psychological and/or cognitive events can be controlled by the content module 30 with respect to, and/or based on, the timing signals from the clock. It will be understood that the further modules 40 are connected to the clock for temporal control of the further modules 40.

Regardless of the sensory modality, the oscillator module 20 may apply the same temporal modulation based on the clock to all modalities. The temporal modulation can be based on a single frequency (i.e., a 40 Hz-modulation targeting gamma oscillations) or at multiple frequencies (i.e. some aspects of the auditory stimulus alternating at 10 Hz and others alternating at 40 Hz). Temporal modulation at the desired frequency can be achieved with a variety of different forms or shapes of the temporal profile of the presented sensory stimulus.

In one aspect, the modulation can achieve a square waveform in which there is a rapid transition between two different states (e.g., changes between low and high volume, pitch, contrast, luminance). The duty cycle of the square waveform can be altered, so that the on-time may represent 50% of the cycle (i.e., half the time in the low state, half the time in the high state) or shorter periods to create impulses (i.e., 10% in the high state, then 90% in the low state) or prolonged on-periods (i.e. 90% in the high state, then 10% in the low state). Alternatively, the modulation can achieve a sinusoidal wave which provides a smooth transition between two different states (e.g., between two levels of zoom or two locations, high and low volume or luminance). Alternatively, the modulation can achieve a sawtooth wave in which there is an abrupt onset followed by a gradual offset or vice versa.

The visual patterns presented by the oscillator module or inducer module 20 may be based on visual stimuli aimed at the early visual cortex, to ensure a visual response. The patterns may be based on patterns seen during visual hallucinations (i.e., due to drugs or migraines) which are thought to represent stable states of large-scale activation. The patterns may be mathematically defined patterns (allowing the parameters of the patterns to be rhythmically modulated) or images or videos. The mathematically defined patterns include fractal patterns, images with multiple axes of reflection (i.e., kaleidoscope images), or geometrically defined patterns (i.e. sinusoidal spirals or grating patterns).

Auditory stimuli presented by the oscillator module or inducer module 20 may be artificial or real recordings of instruments, mathematically generated noise, or any other auditory signal.

The oscillating visual stimuli may be conveyed through modulation of the luminance of a visual stimulus, in which the luminance is dynamically altered such that the visual stimulus transitions between an imperceptible visual stimulus, e.g., a black (no luminance) portion of the display 110 and a full-luminance stimulus. Alternatively, the oscillating visual stimuli may be conveyed through modulation of the contrast of the visual stimulus, in which the visual stimulus transitions between a mid-grey level and to full contrast stimulus. Alternatively, the oscillating visual stimuli may be conveyed through modulation of the saturation of the visual stimulus, in which the visual stimulus transitions between a greyscale image and a full-colour image. Alternatively, the oscillating visual stimuli may be conveyed by colour inversion of the visual stimulus, in which the visual stimulus transitions between a colour-inverted form and a non-colour-inverted form of the visual stimulus. Alternatively, the oscillating visual stimuli may be conveyed through the motion of the visual stimulus on the display 110, i.e., through the representation by a changing set of pixels of the display 110. The visual stimulus may, for instance, be made to zoom, pan, or rotate in a rhythmic manner, such that the visual stimulus is perceived as an oscillating visual stimulus.

Alternatively, the oscillating visual stimulus may be conveyed through rhythmic deformation of mathematically defined texture or shape elements within the visual stimulus such that the texture or shape elements within the display morph or change shape in an oscillating manner at a selected frequency. In one aspect, several of the afore-mentioned modulations may be applied at the same time.

The auditory stimuli may be presented by the oscillator module 20 in an oscillating manner. The oscillating auditory stimulus may be conveyed through modulation of the volume of an auditory stimulus or signal, in which the volume is dynamically altered such that auditory stimulus transitions between a low volume state (i.e., quiet or silence) to a high-volume state (i.e., full loudness) in an oscillatory manner. Alternatively, the oscillating auditory stimuli may be conveyed through modulation of the pitch of the auditory stimulus, in which the auditory pitch is modulated between a high pitch and a low pitch tone.

Alternatively, the oscillating auditory stimuli may be conveyed through the repetition of small auditory chunks which occupy less than one cycle of the desired frequency. These chunks may be like notes, such that each note is played subsequently with the note onsets representing the desired frequency. Alternatively, the chunks may be more complex, such as small chunks of noise or complex waveforms. The onset of auditorily distinct chunks may indicate the beginning of each new auditory cycle at the desired frequency.

The content module 30 includes perceptual, psychological, and/or cognitive events and tasks. The perceptual, psychological, and/or cognitive event or task presented to a user is selected based on a target of the intervention, i.e., a targeted neural circuit. The sensory stimulus strongly activates in the targeted neural circuit the task-related pattern of neural activity associated with the processing of the perceptual, psychological, and/or cognitive event or task. The content module 30 includes, amongst others, visual, auditory, somatosensory, tactile, olfactory, gustatory, or vestibular tasks. Visual tasks of the content module 30 may include simple visual features such as lines or 2-D shapes, or complex objects such as faces or real-life scenes. Auditory stimuli of the content module 30 may include sounds and/or music. The content module 30 may further include a multisensory event like a film-clip. The content module 30 may further include a task, such as a memory task.

In this respect, whether the task presented by the content module 30 a task, or a sensory stimulus is irrelevant. Any task will elicit a particular pattern of neural activity. In the context of the current device and method, the content module 30 is designed to elicit neural activity which can be enhanced by the oscillation module 20. The neural activity may be evoked by the task presented by the content module 30.

The content module 30 presents a selected one of the task depending on what neural activity one wishes to elicit and subsequently to enhance.

The task presented by the content module 30 relates to a task-related aspect of information processing that has been identified to require enhancement. The content module 30 does not refer to a particular stimulus per se, but rather describes the space in which any task may be located so to be enhanced by the oscillatory module 20. The presentation of the stimuli from the oscillatory module 20, as detailed above, may precede the presentation of the tasks from the content module 30, or the stimuli from the oscillatory module 20 may be presented simultaneously with the tasks from the content module 30.

Whereas the oscillating sensory stimulus presented by the oscillator module (inducer module) 20 enhances processing, the task presented by the content module 30 influences the areas in the brain to which such enhancement effects are directed. When the sensory stimulus presented by the oscillation module 20 and the task presented by the content module 30 are presented consecutively, simultaneously, or in a temporally overlapping manner, as detailed above, the effect of the oscillation module 20 and content module 30 operating together is to induce the desired oscillations in the brain to influence the initial brain state so that processing of the task presented by the content module 30 will engage the desired neural circuits stronger than without the sensory stimulus from the oscillation module 20. Additionally, the enhancement of oscillations facilitates neuroplasticity, ensuring that the strong engagement of the desired circuit leads to long-lasting enhancement of this activity.

At the start of each session users may be shown instructions with indicate how they are to engage with the task (content image) when presented (see FIG. 5). These instructions can indicate a passive task (no specific instructions, simply perceive the stimuli presented during the content period) or active tasks (with specific instructions to the user on how to engage with the task during the content period) which may or may not include instructing the user on how to make a response during the content period.

As shown in FIG. 5, the session may comprise multiple ones of the stimulus-task-cycle in which a sensory stimulus and content, e.g., the content image, are displayed. At the start of the session, the user may be presented with an initial set of instructions (generated by the content module) that communicates one or more actions that the user is required to perform when viewing the multiple ones of the stimulus-task-cycle. This task may be passive, in which the user is only required to perceive each stimulus-task-cycle and not perform any action. Alternatively, the task may be active, in which the user is given instructions at the start of the session on what actions the users are required to do when presented with the output from the content module. These instructions may or may not require the user to make a response, i.e., the actions may comprise responses. Examples of active tasks without a required response include instructing the user to which parts of the content image they should pay attention or instructing the user to imagine themselves within the scene depicted by the content image. Examples of active tasks with a required response include instructing the user to make a judgement or decision based on the content image such as responding how positive they find the image or whether they recognise the content they are shown.

The method of this document may be implemented on any suitable device, so long as the device can present the oscillating stimuli from the oscillation module 20 adequately. Users who are adequately instructed on how to use the device and abide the task instructions may then benefit from its use.

### Example: Depression

Depression is a heterogeneous and complex mood disorder that affects a growing number of people every year. It has considerable costs on both the individual's wellbeing and to society. It causes significant economic burdens, it is a leading cause of disability across the world, and the second leading cause of death in 15-29-year-olds (WHO 2020).

The general treatment goals for patients or users with depressive disorders are to reduce the symptoms and ultimately to achieve complete remission and reduce the likelihood of a relapse. However, a large proportion of those who develop the disease do not receive adequate care due to low response rates to antidepressant medication and long waiting times for psychotherapy. For this reason, there is a need to provide depressed people with treatment options that overcome these limitations.

One aspect of healthy functioning, which is altered in depression, is the processing and experience of affective or emotional information, i.e., information regarding one's own and/or others' emotions. Unlike healthy individuals, it is assumed that individuals with depression exhibit preferential processing of mood-congruent information in various cognitive domains such as perception, attention, memory and reward. These affective-specific biases are a characteristic of the disease and play a significant role in the development, maintenance and treatment of depression.

While the focus in diagnosis and treatment of mood-related disorders are typically the aspects related to the negative affective system, aberrant functioning of the positive affective system has been associated with several mood disorders including depression, social anxiety, and post-traumatic stress disorders. These mood disorders are typically identified through biases in behavioural tasks such as approach motivation, processing of reward-related cues and reactivity to rewards.

When applied to depression, the device and method according to the present disclosure focus on strengthening and normalising the function of the positive mood related circuits in the brain. To achieve this, in this aspect of the present disclosure, the content module 30 is configured to display positive visual images, i.e., images evoking positive feelings, to users to provide a high engagement of the positive affective circuits in the users' brains (see FIG. 6). To further enable that positive affective circuits are engaged, users are given the task to score each image presented by the content module 30 according to how positive the image makes the users feel. This task directs the users' attention towards assessing their own affective state and further engages the positive affective circuits of the users' brain. The content module 30 presents the users with a task to judge how positive different images makes them feel and may subsequently provide one or more diverse, positive images to be rated or scored. To further enhance the activation of the users' positive affective circuits, the oscillation module 20 may be configured to present sensory stimuli at the theta and/or the gamma frequencies to increase the strength and synchrony of the theta oscillations and the gamma oscillations in the user's brain. The increased strength and synchrony of the theta and the gamma oscillations enhance the processing of the stimuli from the content module 30 in the positive affective circuits and promote neuroplasticity in the brain to allow for long lasting changes in the brain.

To engage the positive affective areas of the brain across multiple stimuli presentations, a range of positive images with diverse content and themes are used to provide that (over several different images) the only constant factor is the positive content of the images. It may be helpful to ensure that users do not habituate to the positive images. The content module 30 will therefore include a large number (e.g., 20′000 images) of high-resolution (e.g., 1000×1000 pixels) photographs that cover as large a range of themes evoking positive feelings as possible, including but not limited to photographs of people (e.g. a couple walking together, a person laughing, a child playing), natural landscapes (e.g. a green forests, mountains illuminated in the sun, a pristine white sandy beach, a still running creak with colourful pebbles), sky images (e.g. sunsets, sunrises, storms, stars at night, or images of the northern lights), animals (e.g. a dog catching a frisbee, a cat sleeping in the sun, an elephant spraying water, a dolphin leaping from the water, a butterfly landing on a flower), plants (e.g. flowers in bloom, large redwood trees), objects (e.g. an artistic photograph of a coffee and cake, an old fashion camera, a rusty car in an overgrown yard), buildings (e.g. photographs of interesting architecture, bridges or monuments), food (e.g. regional meals, images of food preparation, buffet tables of exotic food, street food vendors cooking) and/or macro images (e.g. close up images of people's eyes, insects climbing up leaves, bees pollinating flowers). Examples of images are shown in FIG. 5.

The images may be validated in web-based surveys to determine their rating or score on a positivity scale with different population demographics to aid in the appropriate selection of image by the content module 30 for a specific user. The content module 30 will then assign the image to a user based on images rated positive by other users with similar demographics.

### Example: Acute or Chronic Pain

The term “green light” according to present disclosure means visible light in the range of wavelengths from about 495 nm to about 570 nm. Preferably, the range of wavelengths may be from about 500 nm to about 565 nm. More preferably, the range of wavelengths may be from about 505 nm to about 545 nm. In one aspect, the wavelength is about 525 nm.

In this aspect of the disclosure, the computing system 10 shown in FIG. 1 is configured for the treatment of pain, e.g., acute pain and/or chronic pain, in particular migraine. In further aspects, the computing system 10 can be deployed for the treatment of mood disorders, major depressive disorder, anxiety disorder, bipolar disorder, substance-related disorders, addictive disorders, alcohol use disorder, substance use disorder, disorder due to use of cannabis or cannabinoids, disorder due to use of nicotine, gambling disorder. The computing device 100 may further be a mobile computing device (portable computing device), such as but not limited to a tablet, etc., or a wearable device, such as but not limited to a smart watch, a head-mounted display, a wristband, a chest strap, fitness tracker etc. As mentioned above, the computing device 100 may be part of, or connected to a cloud environment.

The computing system 10 may comprise a light source. The light source may be configured to emit the green light. The light source may comprise one or more LEDs.

The green light may be guided from the light source towards eyes 610 of the user. The green light may be guided by means of one or more optical fibers or waveguides. The waveguides may be two-dimensional waveguides that are integrated into lenses of goggles, such as VR or AR goggles or a head-mounted display.

In one aspect, the light source is a display 110 or screen 110, e.g., a display of a mobile device. In the aspect shown in FIG. 1, the display 110 may be part of a mobile computing device 100, such as a smartphone or a tablet device. The light source may emit the green light alongside displaying an image or a series of images. The light source may emit the green light as at least one of green flicker, a green tint, and a green frame. In one aspect, the green flicker comprises at least one of light flashes or moving objects or patterns on the display 110.

As described above with reference to FIG. 1, the computing system 10 may comprise one or more sensors 160. In one aspect, the computing device 100 may comprise ones of the one or more sensors 160. In this aspect, the computing device 100 may be configured to be worn close to and/or on the surface of the skin of a user. Other ones of the one or more sensors 160 may be separate from the computing device 100. For instance, the other ones of the one or more sensors 160 may be arranged in a wristband or chest strap that communicates with the computing device 100.

The computing device 100 may be configured to detect, analyze, and transmit information concerning e.g., body parameters, such as physiological parameters, and/or ambient data which allow in some cases immediate biofeedback to the wearer. The body parameters relate to one or more of heart rate, heart rate variability, speech, cadence, number of pedestrial steps walked, skin conductance, electrodermal activity, breathing, respiratory rate, blood pressure, sleep cycle, menstrual cycle, body temperature etc. In another aspect of the disclosure, the computing system 10 may comprise means for the user to interact with the computing system 10, e.g., to provide user feedback to the computing system 10. The computing system 10 may initiate or continue a session of the method according to the present disclosure after receiving the body parameters and/or the user feedback.

The one or more programs 171 may further be programmed, upon execution of the one or more programs 171 by the one or more processors 140, to treat a user suffering from pain, e.g., acute pain and/or chronic pain, in particular migraine. In further aspects, the one or more programs 171 may be programmed, upon execution of the one or more programs 171 by the one or more processors 140, to treat a user suffering from mood disorders, major depressive disorder, anxiety disorder, bipolar disorder, substance-related disorders, addictive disorders, alcohol use disorder, substance use disorder, disorder due to use of cannabis or cannabinoids, disorder due to use of nicotine, gambling disorder.

The computing system 10 may further comprise one or both of a VR-headset or head-mounted display 500. The VR headset may comprise one or both of the display 110 or the one or more LEDs.

During the session, the one or more programs 171, e.g., an inducer module 20 may present to the user sensory stimuli, the content module 30 may present to the user content, and a green light module 40 may provide green light.

The one or more programs 171 may comprise the green light module 40. The green light module 40 may be accessed by the one or more processors 140, e.g., by the system processor. The green light module 30 may be executed by the one or more processors 140, e.g., by the system processor.

The visual sensory stimulus in the form of the alternating pattern may further be of any other color than black, such as red, orange, yellow, green, cyan blue, violet, or combinations thereof.

In one aspect of the invention, the pattern may be of a green color. In this way, the sensory stimulus provided by the inducer module 20 and the green light provided by the green light module 40 may be combined and presented to the user simultaneously. In this case, the green light module 40 and the inducer module 20 may cooperate to provide the visual sensory stimulus and the green light, and the content module 30 provides the content. In this aspect, the green light module and the inducer module 20 may merge to a single module for providing the rhythmic sensory stimulus which comprises the green light.

In one aspect of the disclosure, the green light module 30 may display to the user green light images generated based on green light data, which are stored in the green light data storage 460 and acquired by the green light module 30 via the API 300. In one aspect, the green light data 460 comprise green light images that can be displayed via the display 110.

In yet a further aspect, the green light is provided to the user while the user is engaged in further activities by means of the display 110. The user may be engaged in playing a game, e.g., a video game, or in working, e.g., on a text document, a presentation document, a musical composition, a movie, a visual art image, etc. The game the user may be playing may comprise the task which the user will or is engaged in and the processing of which is to be enhanced. The user may be engaged in daily activities. In this aspect, the user may be provided with the green light in the form of a green tint. At least a portion of the display 110 may have a green tint, e.g., an unused portion of the display 110 or the entire area of the display 110 may be tinted green. Alternatively, or additionally, the green light may be presented to the user in the form of at least one green-colored frame of at least one window of a graphical user interface (GUI) that is displayed on the display 110. The green tint and/or the green frame may be modified by making the green tint and/or the green frame flickering. The flickering of the green tint and/or of the green frame may have a pre-determined frequency, e.g., between 6 and 14 Hz, with which the flickering oscillates or changes periodically. The flickering enables inducing neural oscillations in the brain, e.g., in the frequency range of 6 to 14 Hz, preferably between 8 and 13 Hz, for inducing alpha oscillations or waves.

In one further aspect, following a presentation, during an inducer period of a predetermined length of time, of the auditory and/or the visual sensory stimulus, the green light image is presented during a green light period of a predetermined length of time. In one aspect, the inducer period and the green light period may at least partially be overlapping, i.e., occur simultaneously. In another aspect, the inducer period and the green light period do not overlap, i.e., occur at distinct times. The distinct times do not have any point in time in common. In some respects, the sensory stimuli generated by the inducer module 20 will be presented prior to the presentation of the green light generated by means of the green light module 40. In other instances, there is either a partial or complete overlap such that the green light generated by means of the green light module 40 is presented concurrently with sensory stimuli generated by the inducer module 20.

As shown in FIG. 13, a further method of the present disclosure comprises the step 800 of visually exposing the user to green light; and a step 810 of providing the user with at least one sensory stimulus. As mentioned before, the sensory stimulus may occur before, partially overlapping with, or after exposing the user to the green light by means of the green light module 40. The role of the inducer module 20 may be to influence the brain state of the user by means of inducing neural oscillations, such as alpha waves. Inducing alpha waves enables reducing perception of pain in users suffering from acute pain and/or chronic pain, e.g., by decreasing the likelihood of pain being perceived by the patient. The green light module 40 enables reducing perception of pain by inhibiting pain sensation or reducing sensitivity to pain.

The green light triggers nociceptive (i.e., pain-sensation-inhibiting) effects by enhancing the activity of the opioid system. The green light induces the opioids, which, following exposure to noxious stimuli, prevent reduction of the alpha power or reactivate the alpha power. In addition, the green light increases alpha band activity.

Furthermore, inducing and potentiating the power of alpha-band activity by means of the sensory stimulus exerts analgesic (i.e., pain-relieving) effects by decreasing excitatory responses and the flow of information in the circuit involving the thalamus and the prefrontal cortex. The method according to the disclosure thus has the potential to elicit a synergic analgesic effect by acting simultaneously in two different physiological pathways, which can influence each other and thus potentiate the analgesic effects.

The step 800 of visually exposing the user to green light may be embedded in the daily activities of the user. The step 800 may comprise tinting and/or framing the display 110 green. When the user uses the display 110, the user is exposed to the green light from the green tint and/or the green frame.

The step 800 may comprise showing a green light image that has previously been downloaded via the API 300 from the green light data storage 460, on the display 110.

The step 810 of providing the user with at least one sensory stimulus may be embedded in the daily activities of the user. The step 810 may comprise flickering the green tint and/or the green frame, displayed on the display 110 to expose the user to the green light, at a predetermined frequency. The predetermined frequency may lie in the range of between 0 and 200 Hz, preferably between 6 and 14 Hz, more preferably between 8 and 12 Hz. In one aspect, the predetermined frequency is 10 Hz. A frequency between 6 and 14 Hz or between 8 and 12 Hz enables inducing alpha waves in the brain of the user. The flickering of the green tint and/or green frame combines the step 800 of visually exposing the user to the green light and the step 810 of providing the user with at least one sensory stimulus. In this way, the steps 800 and 810 are performed at least partially simultaneously.

The step 810 of providing the user with at least one sensory stimulus and the step 800 of visually exposing the user to green light may be combined into a single step by exposing the user to flickering green light with a flickering frequency suitable for a sensory stimulus as previously described. In this case, the rhythmic sensory stimulus comprises the green light.

The method further comprises the step 820 of engaging the user in a task such as an image rating task, a memorization task, a reaction time task, a decision-making task, actively attending to a stimulus, a breathing exercise.

If the method of the present disclosure is performed using the VR headset 500, the session of the method may last for a predetermined period lying in the range of between 5 and 35 minutes. During this session, an auditory stimulus having, e.g., a frequency lying in the range of between 6 and 14 Hz, such as 10 Hz, may be presented to the user. Furthermore, during this session, the green light may be provided to the user by means of the display 110. The green light may flicker at a frequency lying in the range of between 6 and 14 Hz, such as 10 Hz. The green light may flicker at the same frequency as the frequency of the auditory stimulus.

In FIGS. 2A-2D, possible temporal profiles of the inducer period and the content period during the session of the method according to the present disclosure are shown (see above). In the aspect of the disclosure, in which the providing of the rhythmic sensory stimuli comprise the exposing of the user to the green light (or the visual sensory stimuli comprise the green light), the inducer period will overlap with a green light period (not shown in FIGS. 2A-2D) during which the user is exposed to the green light. The green-light period is an example of the one or more further periods mentioned above.

In the alternative aspect of the disclosure, in which the user is exposed to the green light in addition to being provided with the rhythmic sensory stimuli, the green light period may overlap at least partially with the inducer period and/or overlap at least partially overlap with the content period.

Alternatively, the green light period may overlap with neither the inducer period nor the content period during the session. For example, the green light period may occur in between the inducer period and the content period without any temporal overlap of the green light period with either the inducer period or the content period. In another example, the green light period may occur after the inducer period and the content period.

In one aspect of the disclosure, auditory alpha entrainment in the step 810 is implemented by means of a beat. A hearing range of the user is normally in a range of auditory frequencies between 20 and 20,000 Hz. Auditory or sound frequencies in the alpha band, i.e., approximately 6 to 14 Hz, are not audible to the human ear. The beat is produced by means of two tones that have slightly different auditory frequencies. By superposition, the two tones generate a beat frequency equal to the difference in the auditory frequencies of the two tones. The beat is perceived as a periodic variation in volume, varying at a frequency equal to the difference of the two auditory frequencies.

In one aspect, the beat may be a binaural beat. In this aspect, the two ears of the user each receive an auditory stimulus, the frequencies of which differ. For example, the left ear may receive an auditory sensory stimulus at a frequency of 445 Hz and the right ear may receive an auditory sensory stimulus at a frequency of 455 Hz. The beat, i.e., the volume of the resulting tone, will in this case have an auditory frequency of 10 Hz.

In another aspect, the beat may be a monaural beat. In this aspect, the ears of the user both receive two auditory signals with different frequencies. For example, the left ear and the right ear both receive a signal at a frequency of 445 Hz and simultaneously a signal at a frequency of 455 Hz. The monaural beat may have a beat frequency lying in the range of 6 to 14 Hz, preferably 8 to 12 Hz, such as 10 Hz, for inducing alpha waves in the brain of the user. The monaural beat may be embedded in music, e.g., in music individually customized for the user. In this way, the user may receive the at least one sensory stimulus in the step 810 during the day. In one aspect, the providing of the user with the monaural beat may be initiated based on the body parameters sensed by the wearable device 100. In another aspect, providing the user with the monaural beat may be initiated based on the ambient parameters. In yet another aspect, the providing of the user with the monaural beat may be initiated based on a desire of the user to initiate the session.

As mentioned before, the human ear does not perceive auditory signals having a frequency below approximately 20 Hz. Therefore, auditory signals in the Alpha frequency range will generally not be audible by the user. The auditory signal may thus comprise both audible and inaudible auditory signals (or sound signals).

The square wave, sinusoidal wave, or sawtooth wave, as described above in connection with the temporal modulations of the rhythmic sensory stimuli by the oscillator module 20, can also be applied to the green light. The green light module 40 may apply similar modulations to the green light.

The green light presented to a user is selected based on a target of the intervention, i.e., a targeted neural circuit. The sensory stimulus induces the neural oscillations in the brain. In particular, a rhythmic or oscillating one of the sensory stimulus enables activation of the neural oscillations in the brain. For example, a sensory stimulus oscillating at a frequency of between 6 and 14 Hz, preferably between 8 and 12 Hz, more preferably of about 10 Hz, induces activation and/or enhancement of alpha waves in the brain of the user.

Whereas the oscillating sensory stimulus presented by the inducer module 20 induces neural oscillations in the brain such as alpha waves, the green light activates the endogenous opioid system. The green light in particular increases expression of enkephalin mRNA and activates p-opioid receptors at the level of the rostral ventromedial medulla (RVM). The opioid system induced by the green light prevents, or causes a recovery from, a reduction of the alpha band activity due to exposure to a noxious stimulus. The green light in addition enhances alpha band activity. The oscillating sensory stimulus and the green light thus enable a synergistic effect by action on distinct physiological pathways that may interact with each other and potentiate the analgesic effects.

The system and method according to the present disclosure are directed at treating, e.g., alleviating and/or curing, acute and/or chronic pain.

In further aspects of the invention, the system and method according to the present disclosure are directed at treating, e.g., alleviating and/or curing, migraine, mood disorders, major depressive disorder, anxiety disorder, bipolar disorder, substance-related disorders, addictive disorders, alcohol use disorder, substance use disorder, disorder due to use of cannabis or cannabinoids, disorder due to use of nicotine, or gambling disorder.

Furthermore, the system and method of the present disclosure aim at preventing any relapse after treatment of migraine, mood disorders, major depressive disorder, anxiety disorder, bipolar disorder, substance-related disorders, addictive disorders, alcohol use disorder, substance use disorder, disorder due to use of cannabis or cannabinoids, disorder due to use of nicotine, or gambling disorder.

### Example: Circadian Rhythm-Related Disorders

According to one aspect of the method of reducing circadian rhythm-related disorders such as NES, the user can be prevented from waking up by providing the user with delta oscillations. According to another aspect, fast sleep re-onset can be induced by encouraging relaxation and sleep, by exposure to rhythmic sensory stimuli of a low frequency, such as theta oscillations. The rhythmic sensory stimuli may be designed to induce delta waves having a frequency in a frequency range of 0 Hz to 4 Hz, to induce theta waves having a frequency in a frequency range of 4 Hz to 8 Hz, to induce alpha waves having a frequency in a frequency range of 8 Hz to 12 Hz, or to induce beta waves having a frequency in a frequency range of 12.5 Hz to 30 Hz. The sensory oscillations designed to induce alpha waves may preferably have a frequency in a low alpha range of between 8 Hz to 10 Hz.

The frequency bands of brain waves have been associated with mental conditions of the user. Delta waves (approx. 0.5-4 Hz) have been associated with deep sleep, healing, pain and stress relief, loss of awareness. Theta waves (approx. 4-8 Hz) have been associated with deep relaxation, meditation, mental imagery, intuition, creativity, insight, REM sleep, dreams, and reduced consciousness. Alpha waves (approx. 8-13 Hz) have been associated with physical and mental relaxation, flow state of mind, stress reduction, and drowsiness. Beta waves (approx. 13-4 Hz) have been associated with wakefulness, alertness, analytical thinking, problem solving, decision making, and activity.

The user can be exposed to morning rhythmic sensory stimuli in the morning configured to stimulate ipRGCs in one or more eyes of the user and/or induce neural activity. The user can further be engaged in a morning task such as doing a rating task, doing a savouring task, answering questions, and/or doing a motivational task, to induce behavioural patterns relating to feeding patterns as well as to engage neural circuits relating to positive mood, arousal, and appetite.

The morning rhythmic sensory stimuli may comprise morning light which may comprise monochromatic blue light and may have a wavelength between 430 and 515 nm, preferably between 450 and 495 nm. The morning light may be polychromatic white light band-pass filtered to comprise light with wavelengths of between 430 and 515 nm, preferably of between 450 and 495 nm, most preferably of about 460 nm. The morning light may be polychromatic white light with high power in the short-wavelength region, with a colour temperature between 5000 K and 27000 K, most preferably between 6500 K and 9500 K. The morning light can be flickering or contain oscillating patterns. The morning light may be configured to stimulate ipRGCs. The morning light may have a power in a spectral range, in which ipRGCs are stimulated, that is configured to stimulate ipRGCs.

The user may further be exposed to evening rhythmic sensory stimuli in the evening. The user may further be exposed to night rhythmic sensory stimuli during nocturnal wakefulness. The evening rhythmic sensory stimuli and the night rhythmic sensory stimuli are configured not to stimulate ipRGCs in the eye of the user. The evening rhythmic sensory stimuli and the night rhythmic sensory stimuli are configured to facilitate relaxation and onset of sleep. The user can further be engaged in an evening task, such as performing a mindfulness task, performing a breathing exercise, maintaining a motivation diary, and/or performing a yoga practice, in the evening and/or during nocturnal wakefulness. The evening task is designed to engage neural circuits relating to positive mood, relaxation, and sleep. The evening task is designed to disengage neural circuits relating to appetite or food craving.

The evening rhythmic sensory stimuli may comprise evening light which may have a power in a spectral range, in which ipRGCs are stimulated, that is reduced in comparison to the morning light. The evening light may comprise polychromatic white light. In one aspect, the evening light can be generated by means of a high-pass filter and has a wavelength of, e.g., equal to or greater than 500 nm. In another aspect, the evening light may comprise polychromatic white light with a low power in a wavelength range of approximately 430 nm to 515 nm or comprise white light having a colour temperature of 1000 K-3000 K. The morning light can be flickering or contain oscillating patterns.

In one aspect of the disclosure, the morning light has an intensity of 10-1000 lux, most preferably between 500-1000 lux, at the application surface level. The morning light may have said intensity when entering an eye of the user or when entering an ear channel of the user.

### Example: Immunity

The method according to the present invention can be used to induce positive mood in peri-vaccination periods to enhance immunological responses to inoculation. The method according to the invention can further decrease stress levels over longer periods of time by increasing positive mood in the preceding or proceeding weeks of vaccine inoculation.

The user can be engaged in a task such as being presented with an image to increase positive mood and relaxation. The positive mood and relaxation are reinforced by providing rhythmic sensory stimuli to the user to induce oscillations in the brain of the user and thereby to enhancing neural activity of a neural circuit associated with the processing of the task. Vaccination responses or immunological responses to other immunological challenging events (infectious diseases, cancer, immunosuppressed state) can thus be optimized. This means both better immune responses (measured objectively with immunological markers and medical event outcomes) and psychological responses (less side-effects and greater patient acceptance).

Potential users may be users to be vaccinated in a vaccination center, users that are frequently diagnosed with acute infectious respiratory illness, users that seek stress or anxiety management solutions, users that are in an immunosuppressed state, and users that are in chemotherapy

### Example of Implementation

One example of the device and method of the present disclosure as applied to the case of depression will now be described. The device includes a mobile phone device with an app installed on the device and a mobile phone VR-headset. The mobile phone device will have a display. The mobile device display 110 and earphones connected to the headset adapter are used to present the stimuli from the oscillator module 20 and the content module 30 to the users. The users can then view the mobile phone display through the mobile phone VR-headset, by placing the phone into the device and viewing the phone display through the lenses of the mobile R headset.

While many mobile phone VR-headsets exist, one example is the VR-SHARK X6 VR Headset sold by Hi-Shock Innovative 3D Technologies e.K. This headset includes a bay to insert and hold a mobile phone, 52 mm aspherical lenses, integrated Bluetooth headphones and the required straps and housing to enable the VR-headset to be firmly and comfortably attached to a user's head. With this setup, the patterns and pictures are presented on the mobile phone in landscape mode as a split screen display, where one image is available to each eye 610. When placed in the mobile phone VR-headset, each eye 610 of the user sees only one of the images through the aspherical lens. This optical transformation increases the perceived size of the images shown on the mobile phone display and, by controlling the image presented to each of the eyes 610, enables the user to fixate on the images as if the images were being presented at a distance of 1 m away. This placement enables the user to comfortably view the images as if they were much further away from the eye 610 than presented, however the placement does not alter the content of display in any way.

The users use a Bluetooth connection to connect the mobile phone headset adapter to the mobile phone device so that the sounds will be played via the earphones in the headset. The users start the application on the mobile phone device and then put the mobile phone device into the mobile phone headset adapter and place the headset on their head while seated in a quiet room. The users adjust the position of the headset on their head by tightening the straps of the headset which pass around the back of their head and secure the device comfortably in place. The users can also adjust the sharpness of the image by scrolling a small wheel on the headset, which adjust the focus of the lenses, and set the volume to a comfortable level.

Each session is composed of multiple repetitions of pairs of stimuli that comprise an inducer audiovisual stimulus (from the oscillatory module 20) followed by a positive image (from the content module 30) in close succession.

Each audiovisual inducer stimulus is shown on the mobile phone display device and heard over the Bluetooth headphones for a period of 5 to 10 seconds. During this period the inducer stimulus will be temporality modulated to the desired frequency, here at theta (4-7 Hz) and/or gamma (32-100 Hz), with the modulation to the visual and auditory patterns occurring in synchrony. The visual component of the audiovisual inducer stimulus is presented on the mobile phone display 110 and perceived through the lenses of the headset, while the auditory component of the audiovisual inducer is presented binaurally through the headphones of the headset which are connected via Bluetooth with the mobile phone.

The audiovisual inducer stimuli are selected from the inducer parameter storage 440, e.g., an internally created and managed database, and the program in the device is coded to pick a selection of these audiovisual stimuli from the inducer module 20 in a specific order. This method is non-invasive and is achieved using non-static audiovisual stimuli from the inducer module 20 presented via the senses to induce the desired oscillations in the brain.

In a pre-determined window of time, i.e., the stimulus-task-cycle, following user perception of the audiovisual inducer stimulus, the activity in the theta or gamma oscillations in the brain are increased. This increase can be measured using brain signal recording devices such as but not limited to an EEG device. Following the presentation of the audiovisual inducer stimulus, the content stimulus is presented. This content stimulus is generated by the content module 30 and comprises a positive image selected from the content data storage 450, e.g., an image database of appropriate photos, and is presented on the mobile phone display for a duration of 5-30 seconds. The user will view the content images with their eyes 610 on the mobile phone display, with the images then being processed by the brain. The content images in the content data storage 450, e.g., the image database, are real-world content photos rated high on positive valence and intended to produce positive thoughts and engage the positive affective neural circuits in the brain of the user. Their purpose is to engage the positive affective brain networks, while the boosting effect of the theta or gamma oscillations is still present in the brain. Thus, the processing of the positive content in the content image is amplified.

The method is repeated several times during a single session. Step 1 involves the inducer pattern and sound followed by the step 2 of displaying a positive image. Such single sessions can then be repeated several times daily and on continued basis for the duration of the intervention. The repeated exposure to positive content images and this processing strengthened with the inducer method will over time gradually result in improved mood in the user. Thus, repeated use of the method and the device are designed to attempt a change in the underlying brain circuits and their network properties that will cause a re-training and strengthening of positive-affect processing of depression patients. It is expected that with regular use, these brain circuits can be trained and strengthened like how neuronal and neuro-muscular connections change with regular mental and physical training.

In addition, the user will have the option to select the content per their preference or based on ratings given to the images that then continually update the algorithm recommending images to the viewer. The part of showing the subject images that the users prefer or are expected to prefer is important because this preference utilises the personal experience and innate memories of the person and their associations of the image content with good times in their life. This also reduces the risk of therapy resistance and related issues, as it can be expected that some content is considered as positive by one person, but another viewer might perceive the same content to be neutral or even negative. Using this personalisation approach, the therapy is tailored to individual needs of the person and is thus close to precision medicine. Finally, by using this method, the individual in need of the intervention becomes a key stakeholder in the process of the treatment, which can potentially make the therapy much more effective in terms of patient outcome.

By presenting participants with positive affective stimuli, the neural circuits that are responsible for processing and representing positive affective information are activated. When this pattern of activation is accompanied by the oscillation module 20, inducing the gamma and theta oscillations immediately prior to the onset of the positive affective stimuli, the strength of this activation is enhanced. This enhanced activation of the positive affective circuits combined with the neuroplasticity promoting effect of the gamma oscillations strengthens the neural connections in the positive affective circuit, making them more likely to be engaged again and helping to rebalance the competition between the under-active positive and over-active negative affective circuits in depression.

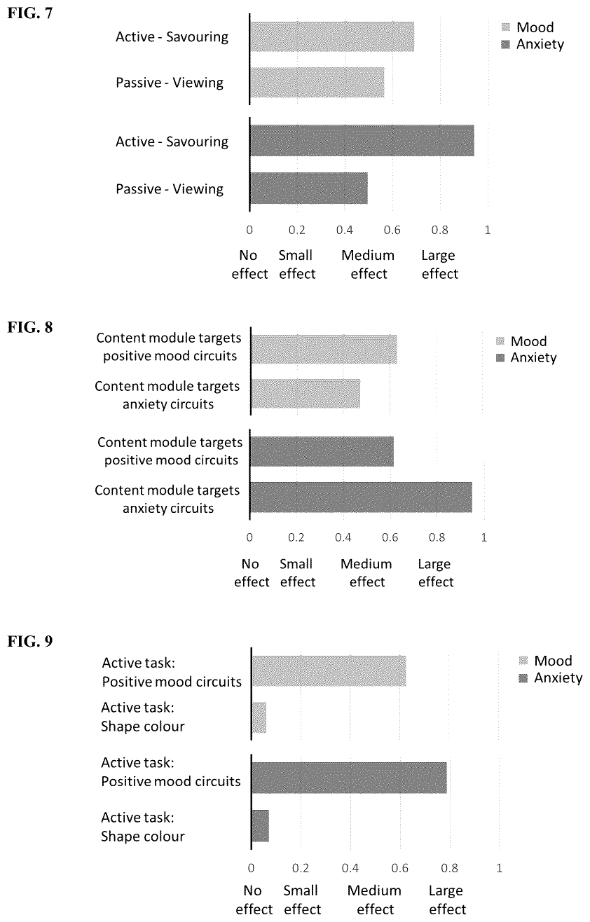

FIG. 7 shows the effect size of the reported mood and anxiety changes after a single 10-minute session with an active (image savouring) or passive (image viewing) task provided by the content module. In two separate experiments, participants viewed 60 images for 10 minutes. The images shown to individuals in both tasks was identical, however task that the user engaged in was differentiated through the delivery of different instructions. In the active task (i.e., Active—Savouring) 44 participants were asked to consciously enjoy and fully immerse themselves into the images (i.e., savouring) and in the passive task (Passive—Viewing) 46 participants were asked to merely pay attention to the images (i.e., passive viewing). As can be seen from this figure depicting effect sizes, positive mood increased similar amount in both tasks, but feelings of anxiety decreased more, when the participants were asked to ‘savour’ the images instead of passively viewing them. Thus, both passive and active tasks can be used to induce positive mood and reduce anxiety.

FIG. 8 shows the effect size of the reported mood and anxiety changes after a single 10-minute session in which the content module 20 was programmed to target positive mood circuits or anxiety circuits. In two separate experiments, participants were asked to rate 60 images in a 10 minute task. In the task designed to induce the positive mood (Content module targets positive mood), 45 participants rated how positive or negative images made them feel on a 7-point Likert scale (from very negative to very positive). In one aspect, the content data storage 450 may comprise an image database of selected images rated in a survey as highly positive (valence). In the other task designed to induce calmer, less anxious state (Content module targets anxiety circuits), 51 participants rated how calm or excited images made them feel on a 7-point Likert scale (from very calm to very excited). In a further aspect, the content data storage 450 may comprise a database of images rated in a survey to have highly positive valence and low arousal. The figure depicting effect sizes shows that rating positive images for valence improved the mood more than rating positive, low arousal images for their excitability. But in terms of reducing anxiety, the presentation of positive, low arousal images and rating them for excitability (calm-excited) was more effective (large effect size) than rating positive images for valence. These experiments demonstrate that by deliberately altering the image and task content (content module), we can effectively boost the effects on the desired target indication (here improving mood or reducing anxiety).

FIG. 9 shows the effect size of the reported mood and anxiety changes after a single 10-minute session in which the content module 30 was optimised to target positive mood circuits or a control task in which users rated the colour of different shapes. In two separate experiments, participants were asked to rate 60 images in a 10 minute task. In the task designed to induce positive mood (Active task: Positive mood circuits), 50 participants rated how images made them feel on a 3-point Likert scale (negative, neutral, positive). In one aspect, the content data storage 450 may comprise an image database of chosen images rated in a survey as highly positive (valence). The other task was designed to act as a control for mood induction procedures (Active task: Shape colour). In this task 60 participants rated the perceived colour of abstract, computer-generated shapes on a 7-point Likert scale (from very yellow to very blue). As can be seen from the figure depicting effect sizes, rating of the positive images significantly improved mood and reduced anxiety (medium-large effect), whereas the shape rating control task did not have effect on mood or anxiety. These findings demonstrate the role of rating positive images (content module) for mood and anxiety effects, and that the active task of rating does not by itself lead to mood improvements and anxiety reduction.

FIG. 14 shows the results of two multi-week online experiments to investigate the effect of method of the disclosure on reducing subclinical depressive symptoms. First, a 6-week online experiment was conducted with a total of 394 participants that were divided into three groups. A first group performed the method according to the present disclosure. A second group was merely presented with the same images as the first group. A third group did not participate in any sessions (control group). Participants in the first and second group performed sessions 4-7 times each week. In each session the participant rated their current mood, calmness and energy level, both before and after the session. The session included 35 repetitions of pairs of inducer stimulus followed by a positive image. The images were to be savored (i.e., immersed into) and rated, on a scale from very negative to very positive, based on how the images made the participant feel. All the participants completed a set of questionnaires on depressive and anxiety symptoms as well as on well-being and lifestyle, at baseline and at the end of weeks 2, 4 and 6. Participants in the first group (n_{baseline}=55, nₚₒₛₜ=44) showed a reduction in depressive symptoms, as measured with the Montgomery-Asberg Depression Rating Scale Self-assessment (MADRS-S), that was statistically significantly different from the second group (n_{baseline}=71, nₚₒₛₜ=57) and the third group (n_{baseline}=65, nₚₒₛₜ=41).

Participants who reported mild-to-moderate subclinical depressive symptoms at baseline (i.e., 8-item Patient Health Questionnaire or PHQ-8≥5) were investigated in a separate experiment. Due to the small sample sizes, a shorter, 2-week online experiment was conducted with an experimental design and setup similar to the 6-week experiment and including improved inducer stimuli. More measures of depressive symptoms were used. Our findings showed that the participants in the first group (n_{baseline}=103, nₚₒₛₜ=103) had a significant decrease of depressive symptoms on all scales used (MADRS-S, Beck Depression Inventory-II, PHQ-8), reduced anxiety symptoms (as measured with the 7-item Generalized Anxiety Disorder scale), and improved calmness and well-being (as measured with a visual analogue scale and the World Health Organization-Five Well-Being Index, respectively). The participants of the third group (n_{baseline}=123, nₚₒₛₜ=79) showed no change in these measures.

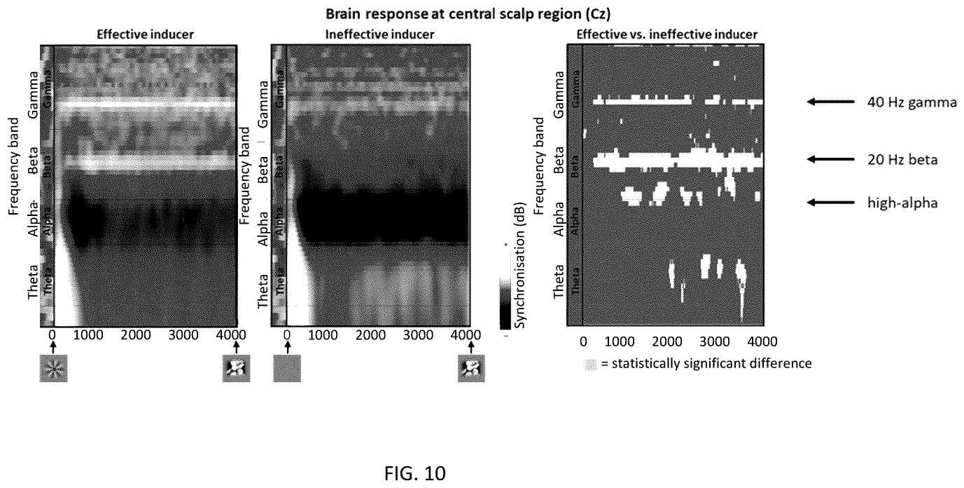

FIG. 10 shows differences in the oscillation synchronization recorded in the brain in response to an effective versus an ineffective inducer. To find a visual inducer that can increase 40 Hz gamma synchronization, complex visual primitives were chosen as visual patterns based on a study comparing different visual stimulus types. The visual patterns were flickered at a rate of 20 Hz to target 40 Hz gamma in the brain (inducing with 40 Hz was not feasible due to technical limitations related to monitor refresh rate). To enhance the effect of this visual inducer, the visual inducer was paired with an auditory inducer. The chosen auditory inducer was a tone, e.g., a monaural tone, at middle C (260 Hz) that was amplitude-modulated with a sine wave function at 40 Hz. The visual inducer and the auditory inducers were combined into dynamic audio-visual inducers. The brain effects were compared between this effective inducer to ineffective inducers, which were composed of a static dot on the screen and/or an unmodulated tone (e.g. a tone, e.g., a monaural tone, at middle C without amplitude modulation, i.e., with constant amplitude). 21 participants were shown the effective inducers, and 17 participants were shown the ineffective inducers. The findings recorded at central location over the scalp (Cz) show the effective inducers to increase synchronization in the intended gamma frequencies (40 Hz), and in the visually induced beta frequency (20 Hz), compared to the ineffective inducers. The increase in gamma synchronization was seen in 90% of the participants, indicating high consistency. These findings show that we can target the chosen 40 Hz gamma frequency and reliably and consistently increase its synchronization with our audio-visual inducers.

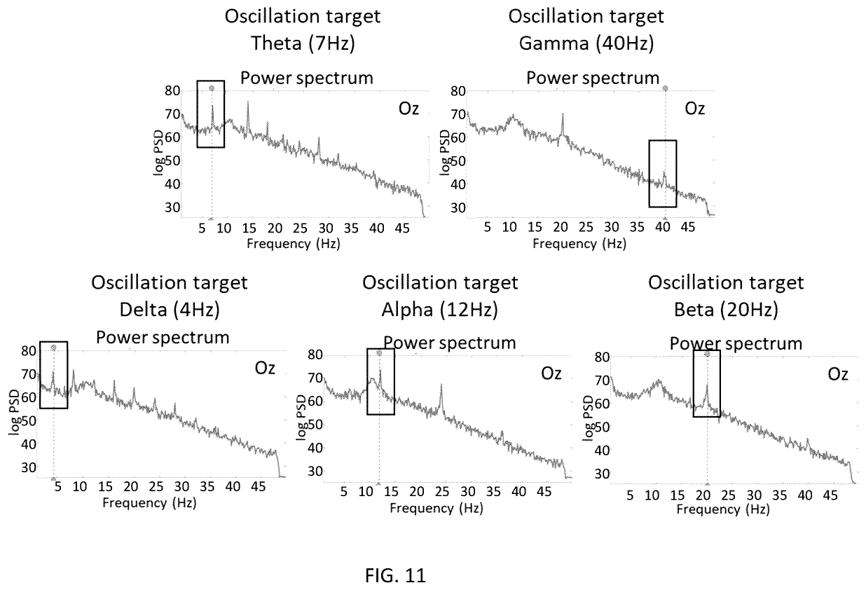

FIG. 11 shows an increase in power at the targeted frequency in the brain for a variety of different inducers targeting different frequency bands via the oscillation module 20 as viewed by the user through the VR-headset 500. The combined mobile phone-EEG experiments involved measuring neurophysiological responses from the participants when the participants were using the session in the mobile phone app and viewing the simple inducers (i.e., screen flickering between black and white at a specific frequency) on the mobile phone screen when the mobile phone was placed inside the headset. The figure demonstrates that when inducing the brain with the visual inducer flickering at a specific frequency, an increase is observed in the amplitude of the power spectrum at the matching frequency in the occipital parts (Oz) of the scalp that are over the early visual processing areas of the brain. This internal test validates the proof of concept of neurophysiological responses to the inducers presented on a mobile phone screen, and that this mobile-EEG setup produces good data. This experiment validates the induction of the oscillations using a common computing device.

FIG. 12 shows EEG data demonstrating that the presence of the inducer, e.g., an audio-visual inducer, enhances the processing of emotional stimuli as evidenced by a faster P3 peak in the ERP signal. It was tested how the audio-visual inducers influence the neural processing of highly positive images. One example of the audio-visual inducers includes complex visual inducer stimulus flickering at a rate of 20 Hz and a monaural tone oscillated with a sine wave function at 40 Hz to increase the 40 Hz gamma synchronization in the brain. The effects of the afore-mentioned audio-visual inducer was compared with a static inducer presenting a static dot and an unmodulated continuous tone. 21 participants were shown the afore-mentioned audio-visual inducer and 17 participants were shown the afore-mentioned static inducer. It was found to show that the afore-mentioned audio-visual inducer enhanced the processing of positive images. In the time-domain, after presentation of the afore-mentioned audio-visual inducer, the P3 event-related potential peaked earlier in comparison to the case of presenting the afore-mentioned static inducer. The P3 event-related potential reflects processes involved in stimulus evaluation and categorization. The earlier occurrence of the P3 event-related potential after presentation of the afore-mentioned audio-visual inducer indicates that the positive images are evaluated and processed faster than after presenting the afore-mentioned static inducer. This effect was consistent across the participants as 90% of the users in the group being presented the afore-mentioned audio-visual inducer show an early P3 event-related potential peak. In conclusion, it was shown that the afore-mentioned audio-visual inducer that increases 40 Hz gamma synchronization in the brain enhances the processing of content images in time-domain.

In another aspect of the present disclosure, the oscillation module 20 could be multimodal, i.e., multisensory, and comprises, e.g., auditory signals and visual signals oscillating in gamma and theta rhythms, respectively. While the visual oscillation module 20 may precede the content module 30 (and be replaced by it) the auditory oscillation module 20 could continue and overlap with the content module 30. In this way, the auditory oscillation module could continue to reinforce the theta and/or gamma oscillations desirable for the task.

In yet a further aspect, parts of the content module 30 itself could be oscillated to reinforce the desirable theta and/or gamma activation. For example, when positive images are displayed as the content module, the luminance, saturation, contrast, or other features of the visual image may be rhythmically manipulated to maintain the oscillatory rhythm established by the oscillation module. In this way, the output from the oscillation module overlaps with the output from the content module.

Regardless of the manifestation, the functional role of the theta and/or gamma oscillations in instantiating enhanced propagations of information processing, the depression-related attenuated processing of positive affect could, in this case, be counteracted by the device and the method.

Specifically, the change in affective information processing by the user is designed to trigger a subsequent clinical improvement in the user's depression. In this sense, the device and method that combine a theta and/or gamma oscillation module with a positive affective content module can provide an effective depression intervention that engages and strengthens the aberrant positive mood circuits in the brain within a practical and pragmatic timeframe.

1. A computing system for treatment of a neural disorder of a user comprising:
one or more processors;
a memory;
one or more output devices;
an oscillator module stored in the memory and adapted to run on the one or more processors, thereby interacting with the output device to produce rhythmic sensory stimuli, perceivable by senses of the user, at at least one predetermined frequency to induce oscillations in a brain of the user and thereby to enhance neural activity of a neural circuit associated with the processing of a task;
a content module stored in the memory and adapted to run on the one or more processors, thereby interacting with the output device to produce content to engage the user in the task.

2. The computer system of claim 1, wherein the one or more output devices comprise at least one of an auditory device, visual device, or tactile device.

3. The computer system of claim 1, wherein the rhythmic sensory stimuli comprise at least one of visual stimuli, auditory stimuli, somatosensory stimuli, olfactory stimuli, gustatory stimuli, and vestibular stimuli.

4. The computer system of claim 1, wherein the content comprises at least one of a visual task, an auditory task, a somatosensory task, an olfactory task, a gustatory task, and a vestibular task.

5. The computer system of claim 1, further comprising measuring devices to measure activity of the brain.

6. A method for treatment of a neural disorder in a user comprising:
providing rhythmic sensory stimuli, perceivable by senses of the user, at at least one predetermined frequency to induce oscillations in a brain of the user and thereby to enhance neural activity of a neural circuit associated with the processing of a task; and
engaging the user in the task.

7. The method of claim 6, wherein the providing of the rhythmic sensory stimuli and the engaging in the task occur within a predetermined window of time.

8. The method of claim 6, wherein the providing of the rhythmic sensory stimuli and the engaging in the task occur at distinct times or at least partially simultaneously.

9. The method of claim 6, wherein the at least one predetermined frequency is selected from the range of between 0 Hz and up to 300 Hz.

10. The method of claim 6, wherein the rhythmic sensory stimuli comprise at least one of visual stimuli, auditory stimuli, somatosensory stimuli, olfactory stimuli, gustatory stimuli, and vestibular stimuli

11. The method of claim 6, wherein the engaging in the task comprises providing at least one of a visual task, an auditory task, a somatosensory task, an olfactory task, a gustatory task, and a vestibular task.

12. The method of claim 6, wherein the engaging in the task comprises providing task-related instructions to the user.

13. The method of claim 6, wherein the providing of the rhythmic sensory stimuli is carried out using one or more output devices.

14. The method of claim 6, wherein the providing of the rhythmic sensory stimuli comprises presenting a sensory stimulus by means of one or more output devices, wherein a first parameter of the presentation is changed at a first one of the at least one predetermined frequency, and a second parameter of the presentation is changed at a second one of the at least one predetermined frequency.

15. The method of claim 6, wherein the engaging in the task comprises at least one of rating a displayed picture, memorisation of one or more facts, measuring a reaction time, making a decision, or actively attending to a stimulus.

16. The method of claim 6, wherein the at least one predetermined frequency lies in the frequency range of alpha oscillations, beta oscillations, delta oscillations, gamma oscillations or theta oscillations.

17. The method of claim 6, further comprising measurement of neural activity.

18. The method of claim 17, wherein variation of the at least one predetermined frequency is dependent on the measured neural activity.

19. The method of claim 6, further comprising:
repeating the step of providing rhythmic sensory stimuli, perceivable by senses of the user, at at least one predetermined frequency to induce oscillations in a brain of the user and thereby to enhance neural activity of a neural circuit associated with the processing of a task; and
repeating the step of engaging the user in the task.

20. The method of claim 19, wherein the repetition takes place over the course of one or more days.

21. Use of the method of claim 6 for the treatment of at least one of depression, including major depressive disorder, dysthymia, disruptive mood dysregulation disorder, and premenstrual dysphoric disorder: anxiety, including separation anxiety disorder, social phobia, agoraphobia, and generalised anxiety disorder; acute stress disorder; adjustment disorder; illness anxiety disorder; cyclothymic disorder; substance/medication-induced depressive anxiety; bipolar disorder, including bipolar I disorder and II disorder; or for the enhancement of immunity.
